(19) United States
(12) Patent Application Publication
(10) Pub. No.: US 2025/0322906 A1
(43) Pub. Date: Oct. 16, 2025

(54) SYSTEMS AND METHODS FOR DISTINGUISHING NUCLEIC ACIDS IN MIXED SAMPLES

(71) Applicant: CareDx, Inc., Brisbane, CA (US)

(72) Inventor: Yi FU, Brisbane, CA (US)

(21) Appl. No.: 19/245,981

(22) Filed: Jun. 23, 2025

Related U.S. Application Data
(63) Continuation of application No. PCT/US2023/085454, filed on Dec. 21, 2023.
(60) Provisional application No. 63/435,153, filed on Dec. 23, 2022.

(51) Int. Cl. G16B 20/10; G16B 20/20

(52) U.S. Cl. CPC G16B 20/10; G16B 20/20

(57) ABSTRACT

Disclosed herein are computer-implemented systems, kits, and methods for outputting an amount of contributor-derived nucleic acids in a biological sample, from a transplant recipient who has received at least two transplants, that comprises nucleic acids from at least three genetically distinct contributors. The amount of contributor-derived nucleic acids may be useful in monitoring the status of a transplant for, e.g., assessing a risk of transplant rejection. In some examples, the at least three genetically distinct contributors may comprise a recipient genomic contributor, a first transplant donor genomic contributor, and a second transplant donor genomic contributor. For example, the systems and methods determine an estimated percentage of the contributor-derived nucleic acids and/or estimated percentage of the fetal-derived nucleic acids.

### CROSS REFERENCE TO RELATED APPLICATIONS

This application is a continuation of and claims priority to International Application No. PCT/US2023/085454, filed on Dec. 21, 2023, which claims the benefit of U.S. Provisional Application No. 63/435,153, filed on Dec. 23, 2022, the contents of each of which are incorporated herein by reference in their entireties for all purposes.

### FIELD OF THE DISCLOSURE

This disclosure relates generally to computer-implemented systems, kits, and methods for determining, without prior genotype knowledge, an amount of contributor-derived nucleic acids in a biological sample from a transplant recipient that comprises nucleic acids from two or more genetically distinct genomic contributors.

### BACKGROUND OF THE DISCLOSURE

Monitoring the health status of transplanted organs, tissues, and cells that a transplant recipient received from a donor using polymorphic markers is particularly complex when the recipient carries additional genomes from genetically distinct contributors, is related to the donor, and/or genotype information, such as single nucleotide polymorphism (SNP) genotype information for identifying which allele belongs to which genomic contributor, is not available. This may be the case in multi-organ transplant scenarios where the transplant recipient received, from one or more donors, at least one organ, tissues, and/or cell transplant, either simultaneously or sequentially, which then become genomic contributors, so that the genetically distinct contributors comprise, for example, in a case where the transplant recipient has received at least two transplants, a recipient genomic contributor, a first transplant donor genomic contributor, and a second transplant donor genomic contributor.

Alternatively, or in addition, the transplant recipient may carry additional genomes from at least two genetically distinct contributors if she became pregnant before or after receiving at least one organ, tissues, and/or cell transplant, with both the fetus and the transplant(s) being contributors, so that the genetically distinct contributors comprise, for example, a maternal genomic contributor, a fetal genomic contributor, and a transplant donor genomic contributor.

Currently, there is an unmet need for monitoring the health of transplanted organs, tissues, and cells in a transplant recipient who carries additional genomes from at least two genetically distinct contributors due to pregnancy and receipt of at least one organ, tissues, and/or cell transplant, or due to receipt of two or more simultaneous or sequential organ, tissues, and/or cell transplants.

### BRIEF SUMMARY OF THE DISCLOSURE

A computer-implemented method of determining an amount of contributor-derived cell-free nucleic acids in a mixed sample, obtained from a transplant recipient, comprising cell-free nucleic acids from at least three genetically distinct contributors, is disclosed. The method comprises: receiving, via a computer or an input function, nucleic acid sequence data from a panel of single nucleotide polymorphisms (SNPs) from the cell-free nucleic acids from the at least three genetically distinct contributors; receiving a genomic relationship among the at least three genetically distinct contributors; determining and grouping minor allele frequency (MAF) information from the panel of SNPs; and determining the amount of contributor-derived cell-free nucleic acids based on the genomic relationship and the MAF grouping. Additionally, or alternatively, in some embodiments, the transplant recipient is a pregnant woman, and the at least three genetically distinct contributors comprise a maternal genomic contributor, a fetal genomic contributor, and a transplant donor genomic contributor. Additionally, or alternatively, in some embodiments, the transplant recipient has received at least two transplants, and the at least three genetically distinct contributors comprise a recipient genomic contributor, a first transplant donor genomic contributor, and a second transplant donor genomic contributor. Additionally, or alternatively, in some embodiments, the amount of contributor-derived cell-free nucleic acids is a percentage of the contributor-derived cell-free nucleic acids in the mixed sample. Additionally, or alternatively, in some embodiments, determining and group MAF information are based on a set of longitudinal samples. Additionally, or alternatively, in some embodiments, the set of longitudinal samples have the same genotype. Additionally, or alternatively, in some embodiments, the panel of SNPs comprises fewer than 500 SNPs. Additionally, or alternatively, in some embodiments, the computer-implemented method further comprises: determining a genotype of one or more of: the transplant recipient, a fetus, or a donor based on the MAF information grouping, wherein the transplant recipient is a pregnant woman. Additionally, or alternatively, in some embodiments, determining and grouping MAF information comprises: reordering the panel of SNPs according to mean or median MAF value; determining the MAF information comprising MAF variation summary statistics in the panel of SNPs; and grouping the panel of SNPs according to the MAF variation summary statistics. Additionally, or alternatively, in some embodiments, determining and grouping MAF information comprises: determining a separation point in the MAF variation summary statistics by determining a local minimum or maximum in a window. Additionally, or alternatively, in some embodiments, the separation point is used to group the SNPs into homozygous and heterozygous genotype groups. Additionally, or alternatively, in some embodiments, determining and grouping MAF information comprises: generating a waterfall plot of the MAF information; grouping the MAF information by segmenting the waterfall plot into groups; and calculating mean MAF values of the groups, wherein the determined amount of contributor-derived cell-free nucleic acids is based on the calculated mean MAF values. Additionally, or alternatively, in some embodiments, determining and grouping MAF information comprises: selecting a first sample comprising a highest mean MAF value among a plurality of samples; selecting a second sample comprising a lowest correlation coefficient associated with the first sample; determining MAF variation summary statistics by subtracting MAF values of the selected first sample and the selected second sample; determining a separation point in the MAF variation summary statistics; and grouping the MAF information based on the separation point. Additionally, or alternatively, in some embodiments, determining and grouping MAF information comprises: selecting an index sample comprising a highest mean MAF value among a plurality of samples; determining an MAF difference between the index sample and each of the plurality of samples; determining MAF variation summary statistics by merging the MAF differences; determining a separation point in the MAF variation summary statistics; and grouping the MAF information based on the separation point. Additionally, or alternatively, in some embodiments, determining and grouping MAF information comprises: selecting a first index sample comprising a highest mean MAF value among a set of high reordered SNPs; selecting a second index sample comprising a highest mean MAF among a set of low reordered SNPs; determining an MAF difference between the first index sample and each of the set of high reordered SNPs; determining an MAF difference between the second index sample and each of the set of low reordered SNPs; determining MAF variation summary statistics by merging the MAF differences; determining a separation point in the MAF variation summary statistics; and grouping the MAF information based on the separation point. Additionally, or alternatively, in some embodiments, the computer-implemented method further comprises: generating a waterfall plot for the mixed sample, wherein the waterfall plot comprises one or more tiers of stairs having one or more steps, and the SNPs of the one or more steps have the same genotype. Additionally, or alternatively, in some embodiments, the transplant recipient received a transplant comprising one or more of: a kidney transplant, a heart transplant, a lung transplant, a liver transplant, a pancreas transplant, a vascularized composite transplant, an intestinal transplant, a stomach transplant, a testis transplant, a penis transplant, an ovary transplant, a uterus transplant, a thymus transplant, a face transplant, a hand transplant, a leg transplant, a bone transplant, a cornea transplant, skin transplant, a heart valve transplant, a blood vessel transplant, or any combination thereof. Additionally, or alternatively, in some embodiments, the mixed sample is a blood sample.

A kit for determining an amount of contributor-derived cell-free nucleic acids in a mixed sample, obtained from a transplant recipient, comprising cell-free nucleic acids from at least three genetically distinct contributors, is disclosed. The kit comprises instructions for: receiving, via a computer or an input function, nucleic acid sequence data from a panel of single nucleotide polymorphisms (SNPs) from the cell-free nucleic acids from the at least three genetically distinct contributors; receiving a genomic relationship among the at least three genetically distinct contributors; determining and grouping minor allele frequency (MAF) information from the panel of SNPs; and determining the amount of contributor-derived cell-free nucleic acids based on the genomic relationship and the MAF grouping. Additionally, or alternatively, in some embodiments, the transplant recipient is a pregnant woman, and the at least three genetically distinct contributors comprise a maternal genomic contributor, a fetal genomic contributor, and a transplant donor genomic contributor. Additionally, or alternatively, in some embodiments, the transplant recipient has received at least two transplants, and the at least three genetically distinct contributors comprise a recipient genomic contributor, a first transplant donor genomic contributor, and a second transplant donor genomic contributor. Additionally, or alternatively, in some embodiments, the amount of contributor-derived cell-free nucleic acids is a percentage of the contributor-derived cell-free nucleic acids in the mixed sample. Additionally, or alternatively, in some embodiments, determining and group MAF information are based on a set of longitudinal samples. Additionally, or alternatively, in some embodiments, the set of longitudinal samples have the same genotype. Additionally, or alternatively, in some embodiments, the panel of SNPs comprises fewer than 500 SNPs. Additionally, or alternatively, in some embodiments, the kit further comprises instructions for: determining a genotype of one or more of: the transplant recipient, a fetus, or a donor based on the MAF information grouping, wherein the transplant recipient is a pregnant woman. Additionally, or alternatively, in some embodiments, determining and grouping MAF information comprises: reordering the panel of SNPs according to mean or median MAF value; determining the MAF information comprising MAF variation summary statistics in the panel of SNPs; and grouping the panel of SNPs according to the MAF variation summary statistics. Additionally, or alternatively, in some embodiments, determining and grouping MAF information comprises: determining a separation point in the MAF variation summary statistics by determining a local minimum or maximum in a window. Additionally, or alternatively, in some embodiments, the separation point is used to group the SNPs into homozygous and heterozygous genotype groups. Additionally, or alternatively, in some embodiments, determining and grouping MAF information comprises: generating a waterfall plot of the MAF information; grouping the MAF information by segmenting the waterfall plot into groups; and calculating mean MAF values of the groups, wherein the determined amount of contributor-derived cell-free nucleic acids is based on the calculated mean MAF values. Additionally, or alternatively, in some embodiments, determining and grouping MAF information comprises: selecting a first sample comprising a highest mean MAF value among a plurality of samples; selecting a second sample comprising a lowest correlation coefficient associated with the first sample; determining MAF variation summary statistics by subtracting MAF values of the selected first sample and the selected second sample; determining a separation point in the MAF variation summary statistics; and grouping the MAF information based on the separation point. Additionally, or alternatively, in some embodiments, determining and grouping MAF information comprises: selecting an index sample comprising a highest mean MAF value among a plurality of samples; determining an MAF difference between the index sample and each of the plurality of samples; determining MAF variation summary statistics by merging the MAF differences; determining a separation point in the MAF variation summary statistics; and grouping the MAF information based on the separation point. Additionally, or alternatively, in some embodiments, determining and grouping MAF information comprises: selecting a first index sample comprising a highest mean MAF value among a set of high reordered SNPs; selecting a second index sample comprising a highest mean MAF among a set of low reordered SNPs; determining an MAF difference between the first index sample and each of the set of high reordered SNPs; determining an MAF difference between the second index sample and each of the set of low reordered SNPs; determining MAF variation summary statistics by merging the MAF differences; determining a separation point in the MAF variation summary statistics; and grouping the MAF information based on the separation point. Additionally, or alternatively, in some embodiments, the kit further comprises instructions for: generating a waterfall plot for the mixed sample, wherein the waterfall plot comprises one or more tiers of stairs having one or more steps, and the SNPs of the one or more steps have the same genotype. Additionally, or alternatively, in some embodiments, the transplant recipient received a transplant comprising one or more of: a kidney transplant, a heart transplant, a lung transplant, a liver transplant, a pancreas transplant, a vascularized composite transplant, an intestinal transplant, a stomach transplant, a testis transplant, a penis transplant, an ovary transplant, a uterus transplant, a thymus transplant, a face transplant, a hand transplant, a leg transplant, a bone transplant, a cornea transplant, skin transplant, a heart valve transplant, a blood vessel transplant, or any combination thereof. Additionally, or alternatively, in some embodiments, the mixed sample is a blood sample.

A system for determining an amount of contributor-derived cell-free nucleic acids in a mixed sample, obtained from a transplant recipient, comprising cell-free nucleic acids from at least three genetically distinct contributors, is disclosed. The system comprises: an interface configured to receive an input; a determination unit configured to: receive, via the interface, nucleic acid sequence data from a panel of single nucleotide polymorphisms (SNPs) from the cell-free nucleic acids from the at least three genetically distinct contributors; receive a genomic relationship among the at least three genetically distinct contributors; determine and group minor allele frequency (MAF) information from the panel of SNPs; and determine the amount of contributor-derived cell-free nucleic acids based on the genomic relationship and the MAF grouping. Additionally, or alternatively, in some embodiments, the transplant recipient is a pregnant woman, and the at least three genetically distinct contributors comprise a maternal genomic contributor, a fetal genomic contributor, and a transplant donor genomic contributor. Additionally, or alternatively, in some embodiments, the transplant recipient has received at least two transplants, and the at least three genetically distinct contributors comprise a recipient genomic contributor, a first transplant donor genomic contributor, and a second transplant donor genomic contributor. Additionally, or alternatively, in some embodiments, the amount of contributor-derived cell-free nucleic acids is a percentage of the contributor-derived cell-free nucleic acids in the mixed sample. Additionally, or alternatively, in some embodiments, determining and group MAF information are based on a set of longitudinal samples. Additionally, or alternatively, in some embodiments, the set of longitudinal samples have the same genotype. Additionally, or alternatively, in some embodiments, the panel of SNPs comprises fewer than 500 SNPs. Additionally, or alternatively, in some embodiments, the determination unit is further configured to: determine a genotype of one or more of: the transplant recipient, a fetus, or a donor based on the MAF information grouping, wherein the transplant recipient is a pregnant woman. Additionally, or alternatively, in some embodiments, the determination unit configured to determine and group MAF information comprises the determination unit configured to: reorder the panel of SNPs according to mean or median MAF value; determine the MAF information comprising MAF variation summary statistics in the panel of SNPs; and group the panel of SNPs according to the MAF variation summary statistics. Additionally, or alternatively, in some embodiments, the determination unit configured to determine and group MAF information comprises the determination unit configured to: determine a separation point in the MAF variation summary statistics by determining a local minimum or maximum in a window. Additionally, or alternatively, in some embodiments, the separation point is used to group the SNPs into homozygous and heterozygous genotype groups. Additionally, or alternatively, in some embodiments, the determination unit configured to determine and group MAF information comprises the determination unit configured to: generate a waterfall plot of the MAF information; group the MAF information by segmenting the waterfall plot into groups; and calculate mean MAF values of the groups, wherein the determined amount of contributor-derived cell-free nucleic acids is based on the calculated mean MAF values. Additionally, or alternatively, in some embodiments, the determination unit configured to determine and group MAF information comprises the determination unit configured to: select a first sample comprising a highest mean MAF value among a plurality of samples; select a second sample comprising a lowest correlation coefficient associated with the first sample; determine MAF variation summary statistics by subtracting MAF values of the selected first sample and the selected second sample; determine a separation point in the MAF variation summary statistics; and group the MAF information based on the separation point. Additionally, or alternatively, in some embodiments, the determination unit configured to determine and group MAF information comprises the determination unit configured to: select an index sample comprising a highest mean MAF value among a plurality of samples; determine an MAF difference between the index sample and each of the plurality of samples; determine MAF variation summary statistics by merging the MAF differences; determine a separation point in the MAF variation summary statistics; and group the MAF information based on the separation point. Additionally, or alternatively, in some embodiments, the determination unit configured to determine and group MAF information comprises the determination unit configured to: select a first index sample comprising a highest mean MAF value among a set of high reordered SNPs; select a second index sample comprising a highest mean MAF among a set of low reordered SNPs; determine an MAF difference between the first index sample and each of the set of high reordered SNPs; determine an MAF difference between the second index sample and each of the set of low reordered SNPs; determine MAF variation summary statistics by merging the MAF differences; determine a separation point in the MAF variation summary statistics; and group the MAF information based on the separation point. Additionally, or alternatively, in some embodiments, the determination unit is further configured to: generate a waterfall plot for the mixed sample, wherein the waterfall plot comprises one or more tiers of stairs having one or more steps, and the SNPs of the one or more steps have the same genotype. Additionally, or alternatively, in some embodiments, the transplant recipient received a transplant comprising one or more of: a kidney transplant, a heart transplant, a lung transplant, a liver transplant, a pancreas transplant, a vascularized composite transplant, an intestinal transplant, a stomach transplant, a testis transplant, a penis transplant, an ovary transplant, a uterus transplant, a thymus transplant, a face transplant, a hand transplant, a leg transplant, a bone transplant, a cornea transplant, skin transplant, a heart valve transplant, a blood vessel transplant, or any combination thereof. Additionally, or alternatively, in some embodiments, the mixed sample is a blood sample.

### BRIEF DESCRIPTION OF THE DRAWINGS

FIG. 1 illustrates an example system for determining the amount of contributor-derived nucleic acids in a biological sample, according to some embodiments.

FIG. 2 illustrates a flowchart of an example general computer-implemented method of determining an amount of contributor-derived nucleic acids in a mixed sample comprising cell-free nucleic acids from at least three genomic and genetically distinct contributors, according to some embodiments.

FIG. 3 illustrates a flowchart of an example computer-implemented method of determining an amount of contributor-derived nucleic acids in a mixed sample comprising cell-free nucleic acids from at least three genomic and genetically distinct contributors, according to some embodiments.

FIG. 4 illustrates an example method for grouping single nucleotide polymorphisms (SNPs) and inferring the SNP genotype for some or all genomic contributors, according to some embodiments.

FIGS. 5A and 5B illustrate example waterfall plots of minor allele frequency (MAF) values for a panel of single nucleotide polymorphisms (SNPs), according to some embodiments.

FIG. 6 illustrates an example waterfall plot of minor allele frequency (MAF) values for a panel of single nucleotide polymorphisms (SNPs), according to some embodiments.

FIG. 7A illustrates an example flowchart of the method for determining minor allele frequency (MAF) information in a panel of single nucleotide polymorphisms (SNPs), according to some embodiments.

FIG. 7B shows an example plot of the minor allele frequency (MAF) variation summary statistics, according to some embodiments.

FIG. 7C illustrates an example minor allele frequency (MAF) waterfall plot and a plot of MAF variation summary statistics that comprise three groups of single nucleotide polymorphisms (SNPs) with distinct genotypes of one of the genomic contributors (transplant donor-1), according to some embodiments of the disclosure.

FIG. 8A illustrates an example method for one-to-one minor allele frequency (MAF) variation summary statistics, according to some embodiments.

FIG. 8B illustrates example minor allele frequency (MAF) waterfall plots for five samples, according to some embodiments.

FIG. 8C illustrates an example minor allele frequency (MAF) variation summary statistics, according to some embodiments.

FIG. 9 illustrates an example method for one-to-all minor allele frequency (MAF) variation summary statistics, according to some embodiments.

FIG. 10A illustrates an example method for all-to-all minor allele frequency (MAF) variation summary statistics, according to some embodiments.

FIG. 10B illustrates an example plots of minor allele frequency (MAF) values for a plurality of samples, according to some embodiments.

FIGS. 11A-11D illustrate example comparisons of a simple minor allele frequency (MAF) variation summary statistic of standard deviation, a one-to-one MAF variation summary statistics, a one-to-one MAF variation summary statistics, and an all-to-all MAF variation summary statistics, according to some embodiments.

FIG. 12 illustrates an example flowchart of the method for determining the amount of contributor-derived cell-free nucleic acids, according to some embodiments.

FIG. 13 illustrates an example device that implements the disclosed system, kits, and methods, according to some embodiments.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to a person of ordinary skill in the art, and the general principles defined herein may be applied to other examples and applications without departing from the spirit and scope of the various embodiments. The various embodiments are not limited to the examples described herein but are to be accorded the scope consistent with the claims. All references cited herein, including patent applications and publications, are hereby incorporated by reference in their entirety.

### Definitions

The terminology used in the description of the various embodiments herein is for the purpose of describing particular embodiments only and is not intended to be limiting. As used in the specification and in the appended claims, the singular forms of “a,” “an,” and “the” include plural referents unless the context clearly dictates otherwise. It will also be understood that the term “and/or,” as used herein, refers to and encompasses any and all possible combinations of one or more of the associated listed items. It will be further understood that the terms “includes,” “including,” “comprises,” and/or “comprising,” when used in this specification, specify the presence of stated features, steps, operations, elements, and/or components, but do not preclude the presence or addition of one or more other features, steps, operations, elements, components, and/or combinations thereof.

The term “sample” or “biological sample,” as used herein, refers to any sample obtained from a transplant recipient including, but not limited to, whole blood, plasma, serum, peripheral blood mononuclear cells, lymph fluid, buccal swabs, saliva, sputum, tears, sweat, ear fluid, bone marrow suspension, urine, feces, lung lavage, semen, vaginal fluid, cerebrospinal fluid, brain fluid, ascites, milk, secretions of the respiratory, intestinal or urinary tract fluids, or tissue and/or cells from a biopsy.

The term “amount,” as used herein, refers to any quantitative value that results from the analysis of nucleic acids, and may represent a relative value or an absolute value.

The term “transplant,” as used herein, refers to transplants of any cell(s), tissue(s), or organ(s) from a donor to a recipient, including combinations thereof. The term “transplant” with respect to a tissue or organ may refer to a whole tissue or organ (e.g., a whole liver) or portions thereof. A “transplant” refers to any organ, tissue, or cell transplant that is transplanted alone or in combination with one or more other organ, tissue, or cell transplants.

The term “organ transplant,” as used herein, encompasses both solid organ transplants and hollow organ transplants and includes, but is not limited to, a kidney transplant, a heart transplant, a lung transplant, a liver transplant, a pancreas transplant, a vascularized composite transplant, an intestinal transplant, a stomach transplant, a testis transplant, a penis transplant, an ovary transplant, a uterus transplant, a thymus transplant, a face transplant, a hand transplant, a leg transplant, a bone transplant, a cornea transplant, skin transplant, a heart valve transplant, a blood vessel transplant, or any combination thereof.

The term “tissue transplant” includes tissue transplants, such as skin tissue, and organ tissue transplants, such as ovarian tissue transplants, kidney tissue transplants, lung tissue transplants, pancreatic tissue transplants, esophageal tissue transplants, spleen tissue transplants, or any combination thereof.

The term “cell transplant” includes cellular transplants, such as pluripotent stem cells, multipotent stem cells, blood-forming stem cells (e.g., hematopoietic stem cells), blood cells (e.g., peripheral blood mononuclear cells), cord blood cells, pancreatic islet cells, skin cells, cardiomyocytes, neurons, dendritic cells, macrophages, lymphocytes, NK cells, NKT cells, B cells, T cells, regulatory T cells, or genetically engineered T cells (e.g., chimeric antigen receptor (CAR) T cells). These include, but are not limited to, cells taken directly from a transplant donor for administration into a transplant recipient, cells taken from a transplant donor and genetically engineered before administration into a transplant recipient, cells taken from a transplant donor and cultured before administration into a transplant recipient, cells taken from a transplant donor and subjected to a manufacturing process before administration into a transplant recipient, and any combination thereof. Cells may also be stored before administration into a transplant recipient (i.e., “off-the-shelf” cells).

As used herein, the term “donor” or “transplant donor” refers to a human or non-human subject that is genetically distinguishable from a transplant recipient, where the “donor” donates organs, tissues, and/or cells for transplantation into the transplant recipient. In some embodiments, the donor is a human subject. In other embodiments, the donor is a non-human subject, such as an animal, e.g., a pig. In some embodiments, a transplant from a human or non-human donor is transplanted into a human transplant recipient. In some embodiments, a transplant from a human or non-human donor is transplanted into a non-human transplant recipient. In some embodiments, a donor and a transplant recipient belong to the same species. In some embodiments, a donor and transplant recipient belong to different species, e.g., the donor is a non-human subject, such as an animal, whereas the transplant recipient is a human subject.

The term “nucleic acid,” as used herein, refers to RNA or DNA and may be linear, circular or branched, single or double stranded, or a hybrid thereof. The term “nucleic acid” also encompasses RNA/DNA hybrids. In some embodiments, the term “nucleic acid” refers to any of DNA, RNA, mRNA, miRNA, double-stranded DNA, single-stranded DNA, single-stranded DNA hairpins, DNA/RNA hybrids, RNA hairpins, and fragments and combinations thereof. In some embodiments, the term “nucleic acid” refers to mitochondrial DNA, cell-free mitochondrial DNA, cellular DNA, or cell-free DNA.

The term “cell-free nucleic acid,” as used herein, refers to a nucleic acid that is present outside of a cell and may be circulating. In some embodiments, cell-free nucleic acids are nucleic acids that are present outside of a cell and circulating in various bodily fluids (e.g., blood, plasma, serum, urine, etc.) of a transplant recipient. In some embodiments, “cell-free nucleic acid” refers to any DNA (“cell-free DNA”), RNA (“cell-free RNA”), mRNA, miRNA, double-stranded DNA, single-stranded DNA, single-stranded DNA hairpins, DNA/RNA hybrids, RNA hairpins, as well as fragments and combinations thereof, present outside of a cell. Cell-free DNA may have originated from various locations within a cell, for example, from nuclear DNA and mitochondrial DNA.

The term “cell nucleic acid” or “cellular nucleic acid,” as used herein, refers to a nucleic acid that is present within a cell. In some embodiments, cell nucleic acids or cellular nucleic acids are nucleic acids that are present within a cell and within various bodily fluids (e.g., blood, plasma, serum, urine, etc.) of a transplant recipient. In some embodiments, cell nucleic acid or cellular nucleic acid refers to any DNA, RNA, mRNA, miRNA, double-stranded DNA, single-stranded DNA, single-stranded DNA hairpins, DNA/RNA hybrids, or RNA hairpins present inside a cell.

The term “polymorphic marker,” as used herein, refers to a locus of polymorphism, e.g., where two or more alternative nucleic acid sequences or alleles occur, due to a change of one or more bases, one or more insertions, one or more repeats, one or more deletions, and variations thereof. A polymorphic marker may also be a locus where one or more bases were modified by methylation. Polymorphic markers may include single nucleotide polymorphisms (SNPs), short tandem repeats (STRs), restriction fragment length polymorphisms (RFLPs), variable number of tandem repeats (VNTR's), hypervariable regions, minisatellites, dinucleotide repeats, trinucleotide repeats, tetranucleotide repeats, simple sequence repeats, and insertion elements.

The term “mixed sample,” as used herein, refers to a biological sample, obtained from a transplant recipient, that comprises nucleic acids from multiple, genetically distinct contributors. For example, a sample obtained from the pregnant transplant recipient of a single transplant may comprise nucleic acids from three genetically distinct contributors, such as nucleic acids derived from the transplant recipient, e.g., “contributor 1,” nucleic acids derived from the transplant, e.g., “contributor 2,” and nucleic acids derived from the fetus, e.g., “contributor 3.” As a further example, a sample obtained from the non-pregnant transplant recipient of two or more simultaneous or sequential transplants from different donors may comprise nucleic acids from three or more genetically distinct contributors, such as nucleic acids derived from the transplant recipient, e.g., “contributor 1,” nucleic acids derived from transplant “A”, e.g., “contributor 2,” and nucleic acids derived from transplant “B,” e.g., “contributor 3,” and so forth. As a further example, a sample obtained from the pregnant transplant recipient of two or more simultaneous or sequential transplants from different donors may comprise nucleic acids from four or more genetically distinct contributors, such as nucleic acids derived from the transplant recipient, e.g., “contributor 1,” nucleic acids derived from the two transplants, e.g., “contributor 2” and “contributor 3,” and nucleic acids derived from the fetus, e.g., “contributor 4,” and so forth.

### Overview

The present disclosure is based, at least in part, on Applicant's development of computer-implemented systems, kits, and methods for determining (including estimating) an amount of contributor-derived nucleic acids in a biological sample, from a transplant recipient, that comprises nucleic acids from two or more genetically distinct contributors, which may be useful in monitoring the status of a transplant with respect to, for example, assessing a risk of transplant rejection. In some embodiments, the status of a transplant may, for example, be classified and monitored based on the determined amount of contributor-derived nucleic acids in a biological sample, from a transplant recipient, that may comprise nucleic acids from at least three genetically distinct contributors, such as the transplant recipient, fetus, and transplant donor. For example, the systems and methods determine an estimated percentage of the contributor-derived nucleic acids and/or estimated percentage of the fetal-derived nucleic acids. In some embodiments, the status of a transplant may, for example, be classified and monitored based on the determined (e.g., estimated) amount of contributor-derived nucleic acids in a biological sample, from a transplant recipient, that may comprise nucleic acids from at least three genetically distinct contributors, such as the transplant recipient, first transplant donor, and second transplant donor.

In some embodiments, the contributor-derived nucleic acids may be cell-free nucleic acids derived from a transplant donor, e.g., contributor-derived cell-free DNA. In some embodiments, the contributor-derived nucleic acids may be cellular (cell) nucleic acids derived from a transplant donor, e.g., contributor-derived cellular (cell) DNA. In some embodiments, the contributor-derived nucleic acids may be cell-free nucleic acids derived from a fetus, e.g., fetus-derived or fetal cell-free DNA. In some embodiments, the contributor-derived nucleic acids may be cell-free nucleic acids derived from a transplant recipient, e.g., transplant recipient-derived cell-free DNA.

In some embodiments, the transplant recipient has received one organ transplant and is pregnant. In some embodiments, the transplant recipient has received one organ transplant and has been pregnant recently, e.g., within the past 1, 3, 6, or 12 months. In some embodiments, the transplant recipient has received one tissue transplant and is pregnant. In some embodiments, the transplant recipient has received one tissue transplant and has been pregnant recently, e.g., within the past 1, 3, 6, or 12 months. In some embodiments, the transplant recipient has received one cell transplant and is pregnant. In some embodiments, the transplant recipient has received one cell transplant and has been pregnant recently, e.g., within the past 1, 3, 6, or 12 months.

In some embodiments, the transplant recipient has received multiple simultaneous or sequential organ transplants from multiple, genetically distinguishable transplant donors and is pregnant. In some embodiments, the transplant recipient has received multiple simultaneous or sequential organ transplants from multiple, genetically distinguishable transplant donors and has been pregnant recently, e.g., within the past 1, 3, 6, or 12 months.

In some embodiments, the transplant recipient has received multiple simultaneous or sequential tissue transplants from multiple, genetically distinguishable transplant donors and is pregnant. In some embodiments, the transplant recipient has received multiple simultaneous or sequential tissue transplants from multiple, genetically distinguishable transplant donors and has been pregnant recently, e.g., within the past 1, 3, 6, or 12 months.

In some embodiments, the transplant recipient may not be pregnant or has not been recently pregnant. In some embodiments, the transplant recipient has received multiple simultaneous or sequential organ transplants from multiple, genetically distinguishable transplant donors. In some embodiments, the transplant recipient has received multiple simultaneous or sequential tissue transplants from multiple, genetically distinguishable transplant donors. In some embodiments, the transplant recipient has received multiple simultaneous or sequential cell transplants from multiple, genetically distinguishable transplant donors. In some embodiments, the transplant recipient has received multiple simultaneous or sequential organ, tissue, and/or cell transplants from multiple, genetically distinguishable transplant donors.

The various embodiments described herein may be carried out without prior genotype knowledge (a pre-determined genotype) from any genomic contributor, such as SNP genotype information for identifying, for a particular SNP, which allele belongs to which genomic contributor. Accordingly, an amount of contributor-derived, cellular or cell-free, nucleic acids in a biological sample, from a transplant recipient, that comprises nucleic acids from two or more genetically distinct contributors may be determined without obtaining, considering, or using prior or predetermined genotype information from the transplant recipient, from any transplant donor, or any other genotype information from any source. Such prior or predetermined genotype information may include, for example, genotype information from the transplant recipient or from any transplant donor across the whole genome or of portions thereof and/or genotype information at the particular polymorphic markers, e.g., selected SNPs, being analyzed (the SNP genotype). In some embodiments, individual genotyping of the transplant recipient may not be performed. In some embodiments, individual genotyping of any genetically distinct contributor, e.g., any transplant donor, may not be performed. In some embodiments, neither the transplant recipient nor any transplant donor may be individually genotyped. In some embodiments, prior or predetermined genotype information from the transplant recipient may not be considered when determining an amount of contributor-derived, cellular or cell-free, nucleic acids in a biological sample, from a transplant recipient, that comprises nucleic acids from two or more genetically distinct contributors. In some embodiments, the amounts of contributor-derived, cellular or cell-free, nucleic acids in a biological sample, from a transplant recipient, that comprises nucleic acids from two or more genetically distinct contributors may be determined without consideration of prior or predetermined genotype information (SNP genotype) from the transplant recipient and without consideration of prior or predetermined genotype information from any transplant donor. In some embodiments, the amount of contributor-derived, cellular or cell-free, nucleic acids in a biological sample, from a transplant recipient, that comprises nucleic acids from two or more genetically distinct contributors may be determined without using any prior or predetermined genotype information from any genetically distinct contributor.

In accordance with the various embodiments described herein, an amount of contributor-derived, cellular or cell-free, nucleic acids in a biological sample, from a transplant recipient, that comprises nucleic acids from two or more genetically distinct contributors can be determined following an experimental (or laboratory) workflow involving extraction of cell-free nucleic acids or cellular nucleic acids from a biological sample obtained from a transplant recipient, targeted amplification and targeted high-throughput sequencing of selected polymorphic loci, e.g., selected SNPs, as described in U.S. patent application Ser. No. 14/658,061, filed Mar. 13, 2015, and U.S. patent application Ser. No. 17/351,040, filed Jun. 17, 2021, respectively, both of which are hereby incorporated by reference in their entirety.

In some embodiments, an amount of contributor-derived, cellular or cell-free, nucleic acids in a biological sample, from a transplant recipient, that comprises nucleic acid from two or more genetically distinct contributors may be determined following the receipt of experimental data, such as sequencing reads, or other data-related information, such as quality control-related data, results, genotype information, SNP mutation rate, and so forth, from a database or other non-experimental source.

In some embodiments, an amount of contributor-derived, cellular or cell-free, nucleic acids in a biological sample, from a transplant recipient, may be a relative value that is expressed as a ratio or percentage of contributor-derived, cellular or cell-free, nucleic acids relative to total, cellular or cell-free, nucleic acids. In some embodiments, for example, where a pregnant transplant recipient has received an organ transplant from a transplant donor, an amount of contributor-derived cell-free nucleic acids, e.g., transplant donor-derived cell-free DNA, may be a relative value that is expressed as a ratio or percentage of contributor-derived cell-free DNA relative to total cell-free DNA, e.g., cell-free DNA including contributor-derived cell-free DNA plus transplant-recipient-derived cell-free DNA plus fetus-derived cell-free DNA. In some embodiments, for example, where a non-pregnant transplant recipient has received two simultaneous organ transplants from two different transplant donors, an amount of contributor-1-derived cell-free nucleic acids, e.g., transplant donor-1-derived cell-free DNA, may be a relative value that is expressed as a ratio or percentage of contributor-1-derived cell-free DNA relative to total cell-free DNA, e.g., cell-free DNA including transplant donor-1-derived cell-free DNA plus transplant donor-2-derived cell-free DNA plus transplant-recipient-derived cell-free DNA.

In some embodiments, an amount of contributor-derived, cellular or cell-free, nucleic acids may be a relative value that is expressed as a ratio or percentage of nucleic acids that are derived from another genetically distinct contributor, such as the ratio or percentage of cell-free DNA derived from genetically distinct contributor “1” relative to cell-free DNA derived from genetically distinct contributor “2” and/or genetically distinct contributor “3,” and so forth. In some embodiments, for example, where a pregnant transplant recipient has received an organ transplant from a transplant donor, an amount of contributor-1-derived cell-free nucleic acids, e.g., transplant donor-derived cell-free DNA, may be a relative value that is expressed as a ratio or percentage of contributor-1-derived cell-free nucleic acids relative to contributor-2-derived cell-free nucleic acids, e.g., transplant donor-derived cell-free DNA relative to fetus-derived cell-free DNA, or vice versa. In some embodiments, for example, where a non-pregnant transplant recipient has received two sequential organ transplants from two different transplant donors, an amount of contributor-1-derived cell-free nucleic acids, e.g., transplant donor-1-derived cell-free DNA, may be a relative value that is expressed as a ratio or percentage of contributor-1-derived cell-free nucleic acids relative to contributor-2-derived cell-free nucleic acids, e.g., transplant donor-1-derived cell-free DNA relative to transplant donor-2-derived cell-free DNA, or vice versa.

In some embodiments, an amount of contributor-derived, cellular or cell-free, nucleic acids in a biological sample, from a transplant recipient, may be an absolute value that results from comparisons with internal standards or reference standards, or adjustments based on the use of internal standards or reference standards. In some embodiments, for example, an amount of contributor-derived cell-free DNA may be an absolute value that results from comparisons with internal standards or reference standards, or adjustments based on the use of internal standards or reference standards, that were added before or after the extraction of cell-free DNA from the transplant recipient's biological sample.

Accordingly, in some embodiments, a sample obtained from the pregnant transplant recipient of a single organ, tissue, or cell transplant may, thus, be analyzed to determine an amount of recipient-derived (e.g., contributor-1-derived), transplant donor-derived (e.g., contributor-2-derived), and/or fetus-derived (e.g., contributor-3-derived) cell-free DNA as a ratio to or percentage of total (recipient-derived, transplant donor-derived, and fetus-derived) cell-free DNA. In other embodiments, using internal standards or reference standards, a sample obtained from the pregnant transplant recipient of a single organ, tissue, or cell transplant may be analyzed to determine an absolute amount of recipient-derived, transplant donor-derived, and/or fetus-derived cell-free DNA.

In some embodiments, a sample obtained from the non-pregnant transplant recipient of two simultaneous or sequential organ, tissue, or cell transplants from different donors may be analyzed to determine an amount of recipient-derived (e.g., contributor-1-derived), transplant donor-1-derived (e.g., contributor-2-derived), and/or transplant donor-2-derived (e.g., contributor-3-derived) cell-free DNA as a ratio to or percentage of total (recipient-derived, transplant donor-1-derived, and transplant donor-2-derived) cell-free DNA. In other embodiments, using internal standards or reference standards, a sample obtained from the non-pregnant transplant recipient of two simultaneous or sequential organ, tissue, or cell transplants from different donors may be analyzed to determine an absolute amount of recipient-derived, transplant donor-1-derived, and/or transplant donor-2-derived cell-free DNA.

In some embodiments, a sample obtained from the pregnant transplant recipient of two or more simultaneous or sequential organ, tissue, or cell transplants from different donors may be analyzed to determine an amount of recipient-derived (e.g., contributor-1-derived), transplant donor-1-derived (e.g., contributor-2-derived), transplant donor-2-derived (e.g., contributor-3-derived), and/or fetus-derived (e.g., contributor-4-derived) cell-free DNA as a ratio to or percentage of total (recipient-derived, transplant donor-1-derived, transplant donor-2-derived, and fetus-derived) cell-free DNA. In other embodiments, using internal standards or reference standards, a sample obtained from the pregnant transplant recipient of two or more simultaneous or sequential organ, tissue, or cell transplants from different donors may be analyzed to determine an absolute amount of recipient-derived, transplant donor-1-derived, transplant donor-2-derived, and/or fetus-derived cell-free DNA.

Changes in the relative or absolute amounts of contributor-derived cell-free DNA, particularly changes in the relative or absolute amounts of transplant donor-derived cell-free DNA over time, may be useful to inform the status of the transplant (or status of each transplant in case of multiple transplants) in the transplant recipient and/or inform the status of the fetus (in case of a pregnant transplant recipient), as well as inform a need to adjust, e.g., reduce, or maintain an immunosuppressive therapy being administered to the transplant recipient. Changes in the relative or absolute amounts of contributor-derived cell-free DNA, particularly changes in the relative or absolute amounts of transplant donor(s)-derived cell-free DNA over time, may also be useful to determine the risk of transplant rejection.

In some embodiments, an amount of contributor-derived, cellular or cell-free, nucleic acids in a biological sample, from a transplant recipient, may be compared to a suitable threshold value or threshold range to obtain information about the status of the one or more organ, tissue, and/or cell transplant, or the fetus. A threshold value or a threshold range may be a predetermined value or predetermined range that indicates the presence or absence of a condition, or the presence or absence of a risk. The threshold value may be a single cut-off value, such as a median or mean, and may be determined from baseline values before the presence or onset of a condition, or presence of a risk, or after a course of treatment. A baseline value may be the amount of contributor-derived nucleic acids, such as transplant donor-derived cell-free DNA, in pre-transplantation samples from a transplant recipient, which would be presumably zero or negligible, but may also indicate a baseline error in the system. A baseline value may also be the amount of contributor-derived nucleic acids, such as fetus-derived cell-free DNA, in pre-pregnancy samples from a transplant recipient, which would be presumably zero or negligible, but may also indicate a baseline error in the system. Once appropriate analysis parameters are selected, determining the amount(s) of contributor(s)-derived nucleic acids, such as the amount of contributor-derived cell-free DNA in a pregnant transplant recipient, in comparison to a suitable threshold value may inform the status of the transplant. Likewise, determining changes in the amounts of contributor-derived nucleic acids, such as changes in the amounts of contributor-derived cell-free DNA, in a pregnant transplant recipient over a period of time can inform the status of the transplant.

In some embodiments, an amount of contributor-derived, cellular or cell-free, nucleic acids may be compared to prior, i.e., previously determined, amounts derived from the same genomic contributor to obtain longitudinal data and longitudinal information about the status of the one or more organ, tissue, and/or cell transplant, for example, in a non-pregnant recipient of multiple simultaneous or sequential transplants, or the fetus. In some embodiments, an amount of contributor-derived, cellular or cell-free, nucleic acids may be compared to prior, i.e., previously determined, amounts derived from the same genomic contributor to obtain longitudinal data and longitudinal information about the status of the organ, tissue, and/or cell transplant, and the fetus, for example, in a pregnant transplant recipient of a single-donor transplant.

Determining the amount of contributor-derived nucleic acids, such as contributor-derived cell-free DNA, in a biological sample from a pregnant transplant recipient of a single-donor organ, tissue, or cell transplant, may be useful for classifying, determining, and/or monitoring the status of the transplant. The status of the transplant may be valuable and informative with regard to a clinical decision by a treating physician or medical expert involving the treatment of the transplant recipient, for example, with respect to the need for adjusting, e.g., increasing, decreasing, changing, or initiating, the immunosuppressive or anti-rejection treatment of the transplant recipient. The methods of the present disclosure may be useful for classifying, determining, and/or monitoring the status of a transplant based on a determined (including estimated) amount of contributor-derived nucleic acids, such as contributor-derived cell-free DNA, in the case of a pregnant transplant recipient of a single-donor organ, tissue, or cell transplant.

Determining the amount of contributor-1-derived nucleic acids, such as transplant donor-1-derived cell-free DNA, and the amount of contributor-2-derived nucleic acids, such as transplant donor-2-derived cell-free DNA, in a biological sample from a transplant recipient of two simultaneous or sequential organ, tissue, and/or cell transplants from different donors, may be useful for classifying, determining, and/or monitoring the status of one or both transplants. The status of the transplant(s) may be valuable and informative with regard to a clinical decision by a treating physician or medical expert involving the treatment of the transplant recipient, for example, with respect to the need for adjusting, e.g., increasing, decreasing, changing, or initiating, the immunosuppressive or anti-rejection treatment of the transplant recipient. The methods of the present disclosure may be used to classify, determine, or monitor the status of one or more transplants based on a determined (including an estimated value) amount of contributor-derived nucleic acids, such as transplant donor-1-derived cell-free DNA and transplant donor-2-derived cell-free DNA.

### Contributor-Derived Nucleic Acids

The methods of the present disclosure involve the analysis of nucleic acids in a biological sample from a transplant recipient to determine the amounts of contributor-derived nucleic acids, such as cell-free DNA, derived from one or more genetically distinct contributors, e.g., transplant donor(s), which are useful to inform the status of the transplant(s) and/or to inform the need to adjust immunosuppressive therapy that is being administered to the transplant recipient. Likewise, determining a change in the amounts of contributor-derived nucleic acids, such as cell-free DNA, derived from one or more transplants, in a transplant recipient over time according to the methods of the present disclosure may be useful to inform the status of the transplant(s) and/or to inform the need to adjust immunosuppressive therapy that is being administered to the transplant recipient.

In accordance with the various embodiments described herein, an amount of contributor-derived, cellular or cell-free, nucleic acids in a biological sample, from a transplant recipient, that comprises nucleic acids from two or more genetically distinct contributors can be determined following an experimental (or laboratory) workflow involving extraction of cell-free nucleic acids or cellular nucleic acids from a biological sample obtained from a transplant recipient, targeted amplification and targeted high-throughput sequencing of selected polymorphic loci, e.g., a panel of single nucleotide polymorphisms (SNPs), which may be selected as described in U.S. patent application Ser. No. 14/658,061, filed Mar. 13, 2015, and U.S. patent application Ser. No. 17/351,040, filed Jun. 17, 2021, respectively, both of which are hereby incorporated by reference in their entirety.

In some embodiments, an amount of contributor-derived, cellular or cell-free, nucleic acids in a biological sample, from a transplant recipient, that comprises nucleic acids from two or more genetically distinct contributors may be determined following the receipt of experimental data, such as sequencing reads, or other data-related information, such as quality control-related data, results, genotype information, SNP mutation rate, and so forth, from a database or other non-experimental source.

### Transplant Recipients and Samples

The methods of the present disclosure involve determining an amount of contributor-derived nucleic acids, e.g., transplant donor-derived cell-free DNA, from a biological sample obtained from the recipient of an organ, tissue, and/or cell transplant from a transplant donor. The transplant recipient may have received one or more transplants, simultaneously or sequentially. Organ transplants may include, for example, a kidney transplant, a heart transplant, a lung transplant, a liver transplant, a pancreas transplant, a vascularized composite transplant, an intestinal transplant, a stomach transplant, a testis transplant, a penis transplant, an ovary transplant, a uterus transplant, a thymus transplant, a face transplant, a hand transplant, a leg transplant, a bone transplant, a cornea transplant, skin transplant, a heart valve transplant, a blood vessel transplant, or any combination thereof, such as heart-lung or pancreas-kidney transplants. The transplant received by the transplant recipient from the donor may also include tissue transplants, e.g., skin tissue, or cell transplants such as pluripotent stem cells, multipotent stem cells, blood-forming stem cells (e.g., hematopoietic stem cells), blood cells (e.g., peripheral blood mononuclear cells), cord blood cells, pancreatic islet cells, skin cells, cardiomyocytes, neurons, dendritic cells, macrophages, lymphocytes, NK cells, NKT cells, B cells, T cells, regulatory T cells, or genetically engineered T cells (e.g., chimeric antigen receptor (CAR) T cells).

Biological samples from the transplant recipient may include, but not be limited to, whole blood, plasma, serum, peripheral blood mononuclear cells, lymph fluid, buccal swabs, saliva, sputum, tears, sweat, ear fluid, bone marrow suspension, urine, feces, lung lavage, semen, vaginal fluid, cerebrospinal fluid, brain fluid, ascites, milk, secretions of the respiratory, and intestinal or urinary tract fluids, or tissue and/or cells from a biopsy.

Some samples obtained from a transplant recipient may comprise cell-free DNA, and the total cell-free DNA present in the sample may be entirely recipient-derived cell-free DNA, or the total cell-free DNA present in the sample may include a mixture of recipient-derived cell-free DNA and transplant(s)-derived cell-free DNA. Some samples obtained from a pregnant transplant recipient may comprise cell-free DNA, and the total cell-free DNA present in the sample may be entirely recipient-derived cell-free DNA, or the total cell-free DNA present in the sample may include a mixture of recipient-derived cell-free DNA, fetus-derived cell-free DNA, and transplant donor(s)-derived cell-free DNA.

Once a sample is obtained, it can be used directly, frozen, or otherwise stored in a condition that maintains the integrity of the cell-free DNA for certain periods of time by preventing degradation and/or contamination with genomic DNA or other nucleic acids. Samples may be taken from a transplant recipient over a period of time. Samples may be taken from a transplant recipient, before and after transplantation, and, in case of a pregnant transplant recipient, before and after pregnancy, at various times and over various periods of time for use in determining an amount of contributor-derived nucleic acids, according to the methods of the present disclosure. For example, samples may be taken from the transplant recipient within days, weeks, and/or months after transplantation, and in daily, weekly, monthly, and/or yearly intervals. In case of a pregnant transplant recipient, samples may be taken from the transplant recipient within days, weeks, and/or months after conception, during pregnancy and after pregnancy, in daily, weekly, and/or monthly intervals. Samples may be taken from the transplant recipient at various alternative times, as clinically useful and/or indicated. In some embodiments, the time period for obtaining samples from a transplant recipient may be within the first few days after transplantation, e.g., to monitor induction therapy. In some embodiments, the time period for obtaining samples from a transplant recipient may be during tapering off the immunosuppressive regimen, a period that occurs during the first 12 months after transplantation. In some embodiments, the time period for obtaining samples from a transplant recipient may be during the initial long-term immunosuppressive maintenance phase, beginning about 12-14 months after transplantation. In some embodiments, the time period for obtaining samples from a transplant recipient may be during the entire long-term maintenance of the immunosuppressive regimen, e.g., any time beyond 12 months after transplantation.

In some embodiments, samples from the transplant recipient, pregnant or non-pregnant, may be obtained about once every week, about once every 2 weeks, about once every 3 weeks, about once every month, about once every two months, about once every three months, about once every four months, about once every five months, about once every six months, about once every year, or about once every two years or more after the initial sampling event. Appropriate timing and frequency of sampling may be determined by one of skill in the art for a given transplant recipient.

### Analysis of Cell-Free Nucleic Acids (DNA) in a Transplant Recipient

The computer-implemented systems, kits, and methods of the present disclosure involve the analysis of contributor-derived nucleic acids, such as cell-free DNA, in a biological sample, from a transplant recipient, that comprises nucleic acids from two or more genetically distinct contributors. In some embodiments, an amount of contributor-derived nucleic acids, such as cell-free DNA, in a biological sample from a transplant recipient may be determined following the receipt of relevant, pertaining experimental data, such as sequencing reads, or other relevant, pertaining data-related information, such as quality control-related data, results, genotype information, SNP mutation rate, and so forth, from a database or other non-experimental source. In some embodiments, an amount of contributor-derived nucleic acids, such as cell-free DNA, in a biological sample from a transplant recipient may be determined following an experimental (or laboratory) workflow involving extraction of cell-free nucleic acids from a biological sample obtained from a transplant recipient, targeted amplification and targeted high-throughput sequencing of selected polymorphic loci, e.g., a panel of single nucleotide polymorphisms (SNPs), which may be selected as described in U.S. patent application Ser. No. 14/658,061, filed Mar. 13, 2015. After cell-free DNA has been extracted or otherwise obtained from a biological sample from a transplant recipient, a panel of polymorphic markers, suitable for differentiating cell-free DNA derived from various genetically distinct contributors, e.g., for differentiating transplant donor(s)-derived cell-free DNA from recipient-derived cell-free DNA, in the cell-free DNA may be analyzed for determining the amounts of contributor-derived cell-free DNA. Various polymorphic markers may be selected for inclusion in the panel to be analyzed, as long as the polymorphic marker panel as a whole is suitable for differentiating cell-free DNA derived from various genetically distinct contributors, e.g., differentiating transplant donor(s)-derived cell-free DNA from recipient-derived cell-free DNA in the cell-free DNA. Polymorphic markers may include, for example, single nucleotide polymorphisms (SNPs), restriction fragment length polymorphisms (RFLPs), short tandem repeats (STRs), variable number of tandem repeats (VNTRs), hypervariable regions, minisatellites, dinucleotide repeats, trinucleotide repeats, tetranucleotide repeats, simple sequence repeats, and insertion elements. Polymorphic markers may comprise one or more bases modified by methylation. The same polymorphic marker panel may be used for each transplant recipient; there is no need to customize polymorphic marker panels to individualize the panel to different transplant recipients. In some embodiments, the polymorphic markers may be SNPs. SNPs may be selected on the basis that they have, for example, an overall population minor allele frequency of >0.4, a target population minor allele frequency of >0.4, the lowest polymerase error rate (in the test system) of the 6 potential allele transitions or transversions, and low linkage on the genome such as, for example, >500 kb distance between SNPs. SNPs to be included in the SNP panel, or in any other polymorphic marker panel, may be those previously identified as being suitable for differentiating between any two unrelated individuals (Pakstis, A. J., Speed, W. C., Fang, R. et al. SNPs for a universal individual identification panel; Hum Genet 127, 315-324 (2010)). The SNP panel may include at least 10, at least 20, at least 50, at least 100, at least 200, at least 500, at least 1,000, or more SNPs.

### Amplification and Sequencing

Upon extraction from a biological sample from a transplant recipient, nucleic acids, such as cell-free DNA, may be amplified and sequenced for downstream analysis, such as for the analysis of a panel of polymorphic markers, e.g., SNPs, from the cell-free DNA. Various protocols for cell-free DNA extraction, amplification, and sequencing using high-throughput sequencing methods, including next-generation sequencing, are known in the art and described in U.S. patent application Ser. No. 14/658,061, filed Mar. 13, 2015, which is hereby incorporated by reference in its entirety.

### SNP Genotype Inference for Some Contributors by Summarizing and Grouping Single Nucleotide Polymorphism (SNP) Minor Allele Frequency (MAF) Signals

The methods of the present disclosure involve a process of summarizing and grouping (including clustering) MAF signals from which, for biallelic SNPs, the SNP genotype for each genomic contributor can be inferred. Since, in accordance with various embodiments of the present invention, a biological sample from a transplant recipient may comprise cell-free nucleic acids, such as cell-free DNA, from two or more genomic and genetically distinct contributors, the genotype at a particular SNP locus, denoted here as GTᵢ, represents (is composed of) a set of all contributors' genotypes at this SNP. For example, for a pregnant transplant recipient, GT_rpᵢ), the genotype of a particular SNP represents a set of the SNP genotypes of the pregnant transplant recipient (GT_rpᵢ), the transplant donor (GT_dnᵢ) and the transplant recipient fetus (GT_ftᵢ) and can be expressed as GTᵢ:=(GT_rpᵢ, GT_dnᵢ, GT_ftᵢ). Since, in some embodiments, the methods of the present invention can be carried out without the prior or predetermined knowledge of any SNP genotype from any contributor, the SNP genotypes are latent variables that may be inferred in the grouping process.

There are different types and levels of SNP signals that may be summarized from various signals and information, such as nucleic acid sequence reads (nucleic acid sequence data or nucleic acid sequence read counts), SNP allele frequency information (in the form of counts, percentage, or ratio), e.g., reference allele frequency and alternative allele frequency (AAF), major allele frequency and minor allele frequency (MAF), and utilized to infer the SNP genotype for any genomic contributor. Such summarized signals, for each SNP, may be summarized read counts for an amplicon, summarized AAF values, summarized MAF values, and so forth.

In some embodiments, minor allele frequency (MAF) values may be summarized as the representative SNP signal and grouped (clustered) to infer one or more contributor's SNP genotype. For example, the MAF value of the i-th SNP (Sᵢ) may be denoted as xᵢ, and the MAFs of a panel of selected SNPs that was sequenced from cell-free DNA obtained from one sample from a transplant recipient post-transplantation may be denotated as X:=(x₁, x₂, . . . , xₙ), with n being the total number of selected SNPs. In embodiments where multiple, longitudinal samples from the same transplant recipient are analyzed, the MAF value of the i-th SNP (Sᵢ) for the j-th sample may be denoted as

$${x}_{i}^{j} ,$$

X^{j} as all the MAF values for the j-th sample, Sᵢ as the collection of MAF values of all samples on the i-th SNP, and an m×n matrix X̅ as the collection of MAF values of all samples on all SNPs, where m is the number of samples:

$${X}^{j} := ( {x}_{1}^{j} , {x}_{2}^{j} , … , {x}_{n}^{j} ) , j = 1 , 2 , … m$$

$${S}_{i} = ( \begin{matrix}{x}_{i}^{1} \\ … \\ {x}_{i}^{m}\end{matrix} ) , i = 1 , 2 , … , n$$

$$X = ( \begin{matrix}{x}_{1}^{1} & … & {x}_{n}^{2} \\ ⋮ & ⋱ & ⋮ \\ {x}_{n}^{m} & … & {x}_{n}^{m}\end{matrix} ) = ( \begin{matrix}{X}^{1} \\ {X}^{2} \\ ⋮ \\ {X}^{M}\end{matrix} ) = ( {S}_{1} , … , {S}_{n} )$$

In some embodiments, MAF values may be summarized from nucleic acid sequence reads and grouped (clustered) to infer one or more contributor's SNP genotype. For example, in the case of a pregnant transplant recipient, where neither of the three contributors' SNP genotypes: the pregnant transplant recipient (GT_rpᵢ), the transplant's donor (GT_dnᵢ), or the transplant recipient's fetus (GT_ftᵢ), are known, at least two of the three contributors' SNP genotypes may be inferred, on some of the sequenced SNPs, through the signals of X̅. Once at least partial contributors' SNP genotypes are inferred, e.g., two out of three contributors' SNP genotypes, three out of four contributors' SNP genotypes, and so forth, one or more particular contributor-derived amount of nucleic acids may be determined, such as, for example, the amount of transplant donor-derived cell-free DNA and/or the amount of fetus-derived cell-free DNA, or the amount of transplant donor-1-derived cell-free DNA and/or the amount of transplant donor-2 derived cell-free DNA.

In some embodiments, a variate of the original signal matrix X̅, denoted as X̅′, may be employed to infer contributors' SNP genotypes In some embodiments, a “waterfall plot” may be used to visually represent X̅′. X̅′ is an m×n matrix with the reordered SNP signals from the original signal matrix X̅:

$${\overline{X}}^{′} = f ( \overline{X} ) = ( {S}_{1}^{′} , … , {S}_{n}^{′} )$$

$${S}_{{i}^{′}}^{′} = ( \begin{matrix}{x}_{{i}^{′}}^{1} \\ … \\ {x}_{{i}^{′}}^{m}\end{matrix} ) , {i}^{′} = 1 , 2 , … , n$$

$$\{ {S}_{1}^{′} , … , {S}_{n}^{′} \} = \{ {S}_{1} , … , {S}_{n} \}$$

In embodiments, where a waterfall plot contains an arbitrary number of samples from a transplant recipient post-transplantation, X̅′ may be the reshuffled collection of the SNP signals S¿ from the original matrix X̅, by reordering (for example, with descending or ascending order) with some summary statistics of the SNP signals.

### Grouping SNP Signals

In some embodiments, contributors' SNP genotypes, or parts thereof, may be inferred by a grouping (including clustering) process of the SNPs' signals Si. In embodiments, where a waterfall plot may be used for visual representation, the grouping (clustering) process may be similar to “segmenting” the data curves or cascades into groups. For the grouping (clustering) of the SNPs' signals Sᵢ, a group label may be assigned to each SNP in a group to indicate the inferred common contributor genotype shared in that group. For example, SNPs with recipient homozygous/heterozygous genotype after grouping (clustering) may be labeled as recipient_homo/recipient_hetero. Subsequently, for example within the recipient_homo group, SNPs may be further assigned to subgroups and labeled, for example, as fetus_homo/fetus_hetero, to indicate the inferred fetus homozygous/heterozygous genotype.

In embodiments of the present invention, various grouping methods may be used. In some embodiments, thresholding may be used as a grouping method if a binary outcome is desired: for a SNP with the summary statistics of y, it belongs to one (first) group if y is above a given threshold T, and to another (second) group if y is below T. Thresholding may, for example, be employed to infer the transplant recipient SNP genotype, using the SNP summary statistics Y=(y₁, . . . , yₙ). yᵢ, for example using the mean or median of a SNP's MAF values, in which i′ is the new location index for SNPs after the reordering, the same as in the reshuffled X̅″̅:

$${GT\_rp}_{i} , = \{ \begin{matrix}homo , if {y}_{{i}^{′}} < T \\ hetero , if {y}_{{i}^{′}} \geq T\end{matrix} , {i}^{′} = 1 , 2 , … , n$$

$${y}_{{i}^{′}} = mean ( {S}_{{i}^{′}} ) , or {y}_{{i}^{′}} = median ( {S}_{{i}^{′}} )$$

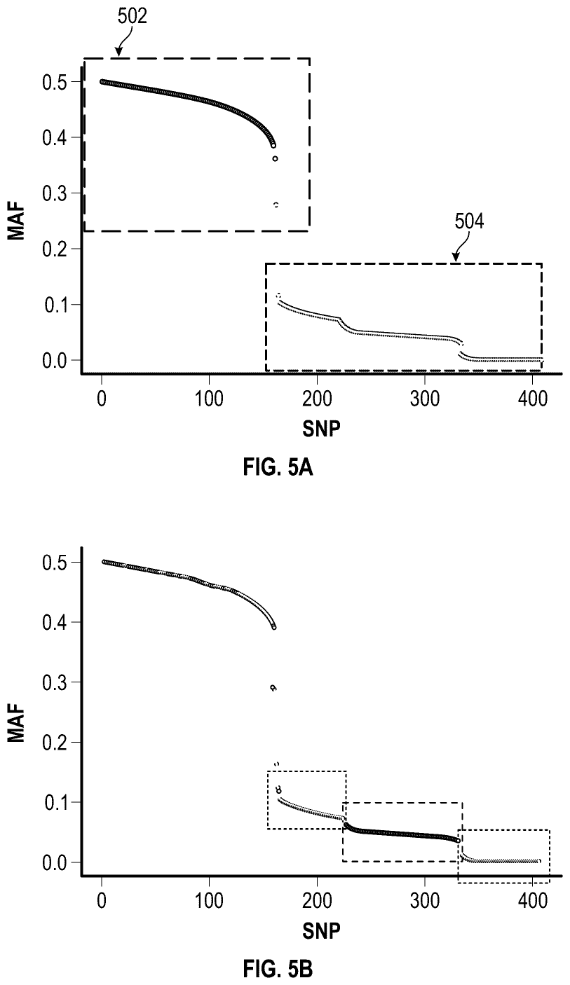

Visually represented, e.g., in a waterfall plot, the genotype of the transplant recipient may be identified in the “first-tier stages” of the plot (for example, as shown in FIG. 5A).

In some embodiments, the grouping process may be based on longitudinal MAF variation, using MAF data or information obtained following the analysis of multiple, longitudinal samples from the same transplant recipient, for example, to infer the genotype of the fetus, of a pregnant transplant recipient, within the transplant recipient homozygous SNP genotype (recipient homo) group. Visually represented, e.g., in a waterfall plot, the genotype of the fetus may be identified in the “second-tier stages” of the plot (for example, as shown in FIG. 5B).

In some embodiments, the SNP's MAF variation summary statistics, denoted as yᵢ, may be chosen through one of the processes (“one-to-one,” “one-to-all,” “all-to-all”) below to summarize the MAF variation and used in grouping of SNPs.

$$Y = ( {y}_{1} , … , {y}_{n} ) = g ( {\overline{X}}^{′} )$$

In some embodiments, the SNP's MAF variation summary statistics yᵢ, may be used to determine a “separation point” P on the reordered location index i′, to group SNPs by comparing their index i′ in the reshuffled X̅′ with P:

$${GT\_ft}_{{i}^{′}} = \{ \begin{matrix}homo , if {i}^{′} \geq P \\ het , if {i}^{′} < P\end{matrix} , {i}^{′} = 1 , 2 , … , n$$

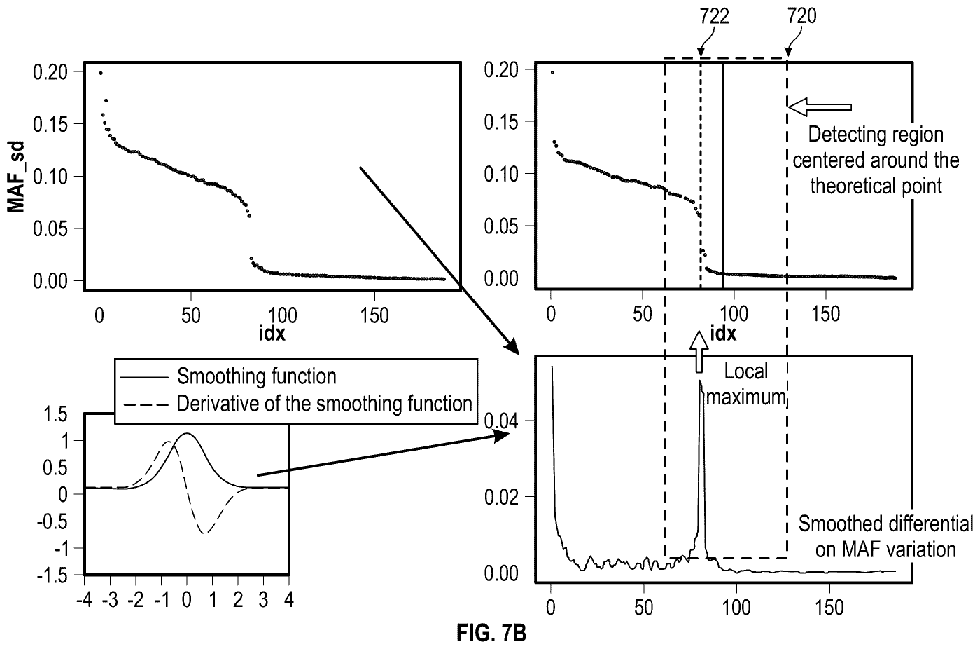

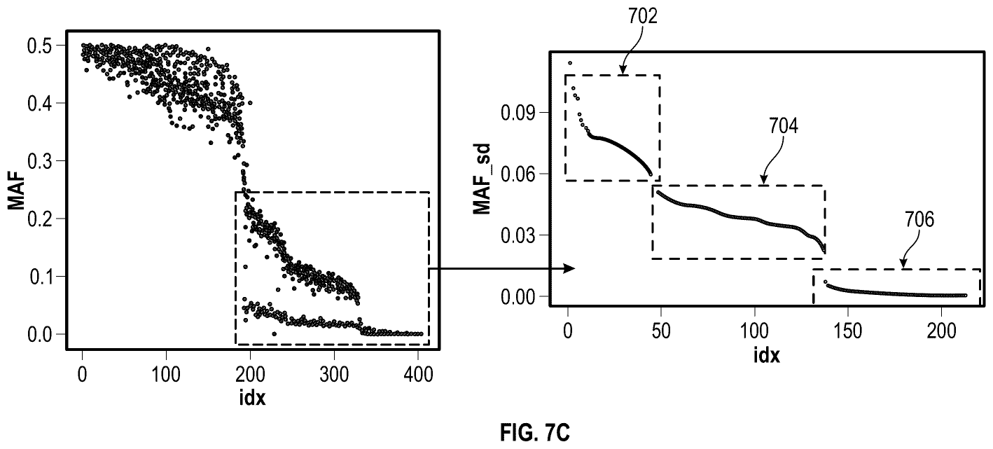

P may be chosen as:

$$P = {arg max}_{{i}^{′}} ( h ( {y}_{{i}^{′}} , Y ) ) ,$$

in which gh may be chosen as a function to reflect the local slope of Y, for example, based on (window-smoothed) sequential difference or rolling difference, as described with respect to FIG. 7B. The above-described embodiments of the present disclosure may be implemented in a variety of ways. For example, some aspects of the embodiments may be implemented using hardware, software, or a combination thereof. When implemented in software, the software code may be executed on any suitable processor or collection of processors, whether provided in a single computer or distributed among multiple computers. Any component or collection of components that perform the functions described above can be generically considered as one or more controllers that control the above-discussed functions. The one or more controllers can be implemented in numerous ways, such as with dedicated hardware, or with general-purpose hardware (e.g., one or more processors) that is programmed using microcode or software to perform the functions recited above.

The implementation of various features of the present disclosure may use at least one non-transitory computer-readable storage medium (e.g., a computer memory, a floppy disk, a compact disk, a tape, etc.) encoded with a computer program (i.e., a plurality of instructions), which, when executed on a processor, performs the above-discussed functions. The computer-readable storage medium may be portable such that the program stored thereon can be loaded onto any computer resource to implement certain aspects of the present disclosure discussed herein. The reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement certain aspects of the present disclosure.

The program can provide a method of determining an amount of contributor-derived, cellular or cell-free, nucleic acids in a biological sample, from a transplant recipient, that comprises nucleic acid from two or more genetically distinct contributors, by accessing experimental data, such as sequencing reads, or other data-related information, such as quality control-related data, results, genotype information, SNP mutation rate, and so forth, from a database or other non-experimental source.

### Determining the Status of a Transplant

The methods of the present disclosure for determining an amount of contributor-derived cell-free nucleic acids, such as cell-free DNA, in a biological sample from a transplant recipient may be used to determine the status of a transplant in a transplant recipient. Amounts of contributor-derived cell-free DNA beyond a suitable threshold value in the transplant recipient as well as changes in the amounts of contributor-derived cell-free DNA over time may be informative with regard to the status of a transplant.

In some embodiments, an amount of contributor-derived cell-free nucleic acids in a transplant recipient above a suitable threshold or an increase in the amounts of contributor-derived cell-free nucleic acids in a transplant recipient over time may be indicative of transplant rejection, a need for adjusting immunosuppressive therapy, immunosuppressive treatment nephrotoxicity, infection, and/or a need for further investigation of the transplant status.

In some embodiments, an amount of contributor-derived cell-free nucleic acids in a transplant recipient below a suitable threshold or a decrease in the amounts of contributor-derived cell-free nucleic acids in a transplant recipient over time may be indicative of transplant tolerance, a need for adjusting immunosuppressive therapy, and/or a need for further investigation of the transplant status.

In some embodiments, no change in the amounts of contributor-derived cell-free nucleic acids in a transplant recipient over time may be indicative of a stable transplant status and/or an opportunity for adjusting, e.g., lowering or discontinuing, immunosuppressive therapy.

### Adjustment of Immunosuppressive Therapy

The methods of the present disclosure for determining an amount of contributor-derived cell-free nucleic acids in a biological sample from a transplant recipient may be used to inform the need to adjust immunosuppressive therapy being administered to the transplant recipient. Immunosuppressive therapy generally refers to the administration of an immunosuppressant or other therapeutic agent that suppresses immune responses to a subject. Exemplary immunosuppressant agents may include, for example, anticoagulents, antimalarials, heart drugs, non-steroidal anti-inflammatory drugs (NSAIDs), and steroids including, for example, Ace inhibitors, aspirin, azathioprine, B7RP-1-fc, β-blockers, brequinar sodium, campath-1H, celecoxib, chloroquine, corticosteroids, coumadin, cyclophosphamide, cyclosporin A, DHEA, deoxyspergualin, dexamethasone, diclofenac, dolobid, etodolac, everolimus, FK778, feldene, fenoprofen, flurbiprofen, heparin, hydralazine, hydroxychloroquine, CTLA-4 or LFA3 immunoglobulin, ibuprofen, indomethacin, ISAtx-247, ketoprofen, ketorolac, leflunomide, meclophenamate, mefenamic acid, mepacrine, 6-mercaptopurine, meloxicam, methotrexate, mizoribine, mycophenolate mofetil, naproxen, oxaprozin, Plaquenil, NOX-100, prednisone, methyprenisone, rapamycin (sirolimus), sulindac, tacrolimus (FK506), thymoglobulin, tolmetin, tresperimus, U0126, and antibodies including, for example, alpha lymphocyte antibodies, adalimumab, anti-CD3, anti-CD25, anti-CD52 anti-IL2R, and anti-TAC antibodies, basiliximab, daclizumab, etanercept, hu5C8, infliximab, OKT4, and natalizumab.

In some embodiments, adjustment of immunosuppressive therapy may include changing the type or form of immunosuppressant or other immunosuppressive therapy being administered to the transplant recipient. In some embodiments where the transplant recipient is not receiving immunosuppressive therapy, the methods of the present disclosure may indicate a need to begin administering immunosuppressive therapy to the transplant recipient.

### Additional Analyses

The methods of the present disclosure may be performed in addition to, or in connection with, other analyses of samples from a transplant recipient to determine an amount of contributor-derived cell-free nucleic acids, such as cell-free DNA, determine the status of a transplant in a transplant recipient, and/or inform the need to adjust immunosuppressive therapy being administered to the transplant recipient.

In some embodiments, nucleic acids extracted from a biological sample of a transplant recipient may also be tested for the presence and/or quantity of infectious agents, such as viruses, bacteria, fungi, parasites, etc. In some embodiments, nucleic acids extracted from a biological sample of a transplant recipient may be tested for the presence and/or quantity of infectious agents that are commonly encountered following organ transplantation. Infectious agents that may be tested for include, but are not limited to, viruses, such as Cytomegalovirus, Epstein-Barr virus, Anelloviridae, and BK virus; bacteria, such as Pseudomonas aeruginosa, Enterobacteriaceae, Nocardia, Streptococcus pneumonia, Staphylococcus aureus, and Legionella; fungi, such as Candida, Aspergillus, Cryptococcus, Pneumocystis carinii; or parasites, such as Toxoplasma gondii.

In some embodiments, the results of the tests for the presence and/or quantity of infectious agents may be used to determine the status of infection in the transplant recipient. In some embodiments, the results of the tests for the presence and/or quantity of infectious agents may be used to inform the need to adjust immunosuppressive therapy being administered to the transplant recipient, such as to lower or change immunosuppressive therapy once the presence and/or particular quantity of infectious agents is confirmed.

The computer-determined status of a transplant may be provided by way of a medical analysis tool that is readily accessible to a physician or medical expert. The medical analysis tool may display the status of a transplant on, e.g., a user interface, a report printout, etc. The physician or medical expert may use the computer-determined status in addition to, or instead of, the physician's or medical expert's assessment of the status of the transplant. The computer-determined status may be provided to the physician or medical expert in the form a determined (estimated) amount of donor(s)-derived cell-free DNA and/or risk of transplant rejection. For example, the medical analysis tool may output the determined (estimated) amount of donor(s)-derived cell-free DNA (0.3%) or a numerical value that indicates, for example, risk of transplant rejection. The computer-determined status may be used by the physician or medical expert as a guide for treatment options, monitoring protocols, and/or clinical diagnosis.

Various techniques and process flow steps will be described in detail with reference to examples as illustrated in the accompanying drawings. In the following description, numerous specific details are set forth in order to provide a thorough understanding of one or more aspects and/or features described or referenced herein. It will be apparent, however, to a person of ordinary skill in the art that one or more aspects and/or features described or referenced herein may be practiced without some or all of these specific details. In other instances, well-known process steps and/or structures have not been described in detail in order to not obscure some of the aspects and/or features described or referenced herein.

In the following description of examples, reference is made to the accompanying drawings which form a part hereof, and in which, by way of illustration, specific examples are shown that can be practiced. It is to be understood that other examples can be used, and structural changes can be made without departing from the scope of the disclosed examples.

### Example Systems and Methods for Determining an Amount of Contributor-Derived Nucleic Acids in a Biological Sample From a Pregnant Transplant Recipient

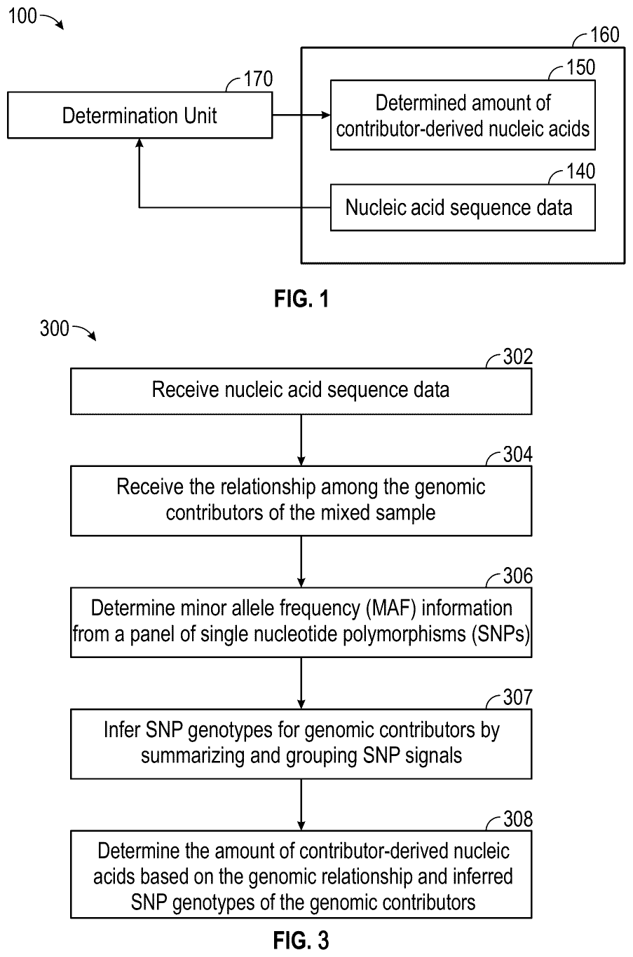

FIG. 1 illustrates an example system 100 for determining the amount of contributor-derived nucleic acids in a biological sample, obtained from the pregnant recipient of an organ transplant (or from a transplant recipient that has received a plurality of transplants), that comprises a mixture of cell-free DNA derived from genetically distinct contributors, e.g., recipient-derived cell-free DNA, fetus-derived cell-free DNA, and transplant donor-derived cell-free DNA.

System 100 may include an interface 160 and a determination unit 170. Various embodiments of the system may include some or all of the components shown in the figure, or other components not shown in the figure. The system 100 may be, for example, a medical analysis tool. A treating physician or medical expert may use the medical analysis tool to help monitor the status of a transplant in a transplant recipient, as well as monitor or evaluate an adjustment to an immunosuppressive therapy administered, or to be administered, to a transplant recipient. The methods of the present disclosure may be used to classify, determine, or monitor the status of a transplant based on the determined amount of contributor-derived nucleic acids in a biological sample, obtained from a transplant recipient, that comprises a mixture of cell-free DNA that is derived from genetically distinct contributors.

In some embodiments, the interface 160 may be used to receive information and/or data obtained following analysis of a mixed sample, such as nucleic acid sequence data 140, or generally input data, and/or provide one or more outputs to the treating physician or medical expert. In some embodiments, the interface 160 may receive the nucleic acid sequence data 140, or input data, via a computer or an input function. In some embodiments, the nucleic acid sequence data 140, or input data, may be, e.g., information from laboratory tests obtained from a blood and/or urine sample of the transplant recipient. Additionally, in some embodiments, the nucleic acid sequence data may comprise relationship information between the genetically distinct contributors. In some embodiments, the interface 160 may output the determined amount of contributor-derived nucleic acids 150 of the sample and/or the status of the transplant. The determined amount of contributor-derived nucleic acids 150 provided by the medical analysis tool may be a percentage of contributor-derived nucleic acids in the biological sample, obtained from a transplant recipient, that comprises a mixture of cell-free DNA derived from genetically distinct contributors. Additionally, or alternatively, the interface 160 may provide an output indicative of the status of the transplant. For example, the interface 160 may output a numerical value that indicates a risk (e.g., high risk, low risk, no risk, etc.) of transplant rejection based on the determined amount of contributor-derived nucleic acids 150.

The determination unit 170 may analyze the nucleic acid sequence data 140 and determine the amount of contributor-derived nucleic acids and/or the status of the transplant. The determination unit 170 may determine the amount of contributor-derived nucleic acids by inferring the SNP genotype for all genomic contributors or for only some contributors by summarizing and grouping (clustering) SNP minor allele frequency (MAF) signals, that were obtained from analyzing a panel of selected SNPs on cell-free nucleic acids from a single sample from the transplant recipient post-transplantation or from multiple, longitudinal samples that were taken from the same transplant recipient over a period of time (days, weeks, months) post-transplantation. In some embodiments, the determination unit 170 may determine a risk of transplant rejection based on the determined amount of contributor-derived nucleic acids 150.

In some embodiments, system 100 may be a kit that may be used, for example, for post-transplant monitoring, according to one or more methods disclosed herein. In some embodiments, the kit may comprise reagents, controls, instructions for use, and/or software instructions for analysis of contributor-derived nucleic acids, such as transplant donor-derived cell-free DNA. In some embodiments, the kit may comprise reagents sufficient to analyze a single sample. In some embodiments, the kit may comprise reagents sufficient to analyze several samples. In some embodiments, the kit may include instructions to specify target values. In some embodiments, the kit may include control materials that may be used in conjunction with the reagents and instructions provided in the kit. In some embodiments, the kit may include instructions for use in accordance with one or more methods disclosed herein. In some embodiments, the kit may comprise instructions for accessing a computer, such as a database, to retrieve data, such as, for example, nucleic acid sequence data. In some embodiments, the kit may comprise reference information, such as scientific literature references, comparative references, package insert materials, clinical trial results. In some embodiments, the kit may include instructions and specifications for input material quality or input preparation methods.

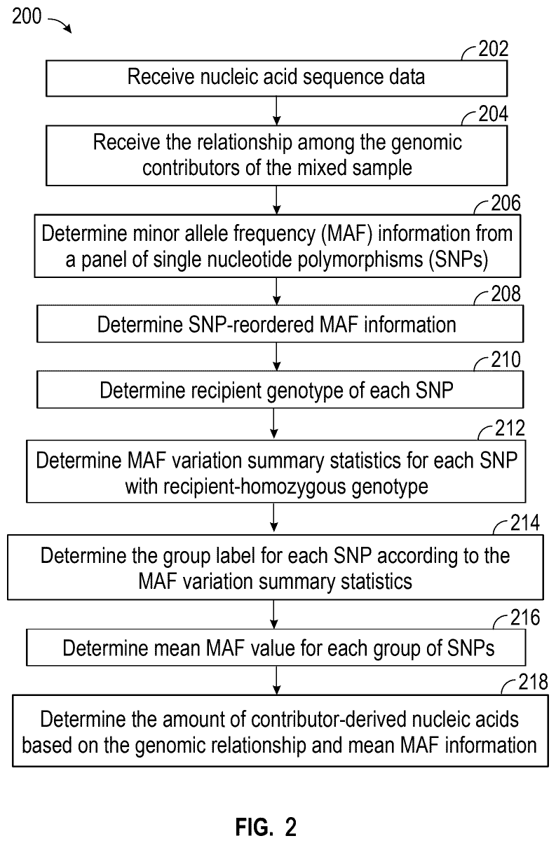

FIG. 2 illustrates a flowchart of an example general computer-implemented method of determining an amount of contributor-derived nucleic acids in a mixed sample comprising cell-free nucleic acids from at least three genomic and genetically distinct contributors, according to some embodiments. Method 200 may comprise step 202, where the system 100 (including its determination unit 170) may receive information and/or data obtained following analysis of a mixed sample, obtained from a transplant recipient post-transplantation. In some embodiments, the information and/or data may be nucleic acid sequence data 140 (reads) that were obtained following sequencing the cell-free nucleic acids from the mixed sample, or general input data.

In some embodiments, the analysis of a mixed sample may comprise an experimental (or laboratory) workflow involving extraction of cell-free nucleic acids from a biological sample, obtained from a transplant recipient, that comprises cell-free nucleic acids, such as cell-free DNA, from at least three genetically distinct contributors; targeted amplification and targeted high-throughput sequencing of selected polymorphic loci, e.g., a panel of single nucleotide polymorphisms (SNPs), which may be selected as described in U.S. patent application Ser. No. 14/658,061. In some embodiments, the analysis of a mixed sample may comprise the receipt of experimental data, such as nucleic acid sequence data 140 (reads), and/or other data-related information, such as quality control-related data, results, genotype information, and so forth, from a database or other non-experimental source. In some embodiments, the experimental data, such as nucleic acid sequence data 140 (reads), and/or other data-related information, such as quality control-related data, results, genotype information, may be received via a computer or an input function. For example, a treating physician or medical expert may provide general input data including, but not limited to, experimental data, such as nucleic acid sequence data 140 (reads) on cell-free nucleic acids from the mixed sample, other data-related information, such as quality control-related data, results, genotype information as input using the interface 160. Additionally, or alternatively, a computer (e.g., a database) may provide general input data including, but not limited to, experimental data, such as nucleic acid sequence data 140 (reads) on cell-free nucleic acids from the mixed sample, other data-related information, such as quality control-related data, results, genotype information, and so forth.

In step 204, a determination unit 170 of the system may receive the genomic relationship among the (genetically distinct) genomic contributors. For example, the mixed sample may be from a pregnant transplant recipient who received a transplant from the fetus' father, so the relationship comprises a mother-child-father relationship. As another example, the relationship comprises a mother-child-paternal aunt relationship if the mixed sample is from a pregnant transplant recipient who received a transplant from the fetus' paternal aunt. Other non-limiting example relationships comprise mother-child-maternal grandmother, mother-child- maternal grandfather, mother-child-paternal grandmother, mother-child-paternal grandfather, mother-child-paternal uncle, mother-child-sibling, mother-child-unrelated donor, etc.

In step 206, a determination unit 170 of the system may determine (and group) minor allele frequency (MAF) information (MAF values or MAF variations) from a panel of single nucleotide polymorphisms (SNPs). Step 208 may comprise determining SNP-reordered MAF information, and step 210 may comprise determining the recipient genotype of each SNP.

In step 212, the system 100 may determine MAF variation summary statistics for each SNP with recipient-homozygous genotype. In step 214, the system 100 may determine the group label for each SNP according to the MAF variation summary statistics.

Step 216 of method 200 may comprise determining the mean MAF value for each group of SNPs. Then, in step 218, the amount of contributor-derived nucleic acids is determined. The amount of contributor-derived nucleic acids may be determined based on the genomic contributor relationship (e.g., received in step 204) and mean MAF information.

FIG. 3 illustrates a flowchart of an example computer-implemented method of determining an amount of contributor-derived nucleic acids in a mixed sample comprising cell-free nucleic acids from at least three genomic and genetically distinct contributors, according to some embodiments. Method 300 may comprise steps 302, 304, and 306, which may be similar to steps 202, 204, and 206 of FIG. 2.

In step 307, the determination unit 170 may infer SNP genotypes for each genomic contributor or some of the genomic contributors by summarizing and grouping a panel of sequenced SNPs from the cell-free nucleic acids from the mixed sample (discussed in more detail above).

In step 308, a determination unit 170 of the system may determine the amount of contributor-derived nucleic acids based on the genomic contributor relationship and the inferred SNP genotypes of the genomic contributors. For example, the determined amount of contributor-derived nucleic acids may be the percentage of the nucleic acids that is transplant donor-derived, such as transplant donor-derived cell-free DNA.

In some embodiments, based on the determined amount of contributor-derived nucleic acids, the system may determine the risk that transplant rejection in the transplant recipient will occur. The determined amount of contributor-derived nucleic acids (from step 308) or the risk of transplant rejection, for example, may be displayed on a computer screen (e.g., interface 160) or described in a report. Additionally, or alternatively, in some embodiments, the system may use the determined amount of contributor-derived nucleic acids to assess the health conditions of the fetus. At a given stage of pregnancy, the fetus cell-free DNA fraction is expected to fall within a target range. If the determined amount of contributor-derived nucleic acids corresponds to a fetus cell-free DNA fraction that is outside of this range, the system 100 may display this or corresponding information on, e.g., a user interface, a report printout, etc. For example, the system 100 may suggest to the physician or medical expert that the fetus may have a potential health issue that should be further investigated.

Although the descriptions and figures show particular steps of the method occurring in a particular order, the steps of the method may occur in other orders not described or shown. Additionally, or alternatively, embodiments of the disclosure may include performing all, some, or none of the steps of method 300, where appropriate. Furthermore, although certain components, devices, or systems are described as carrying out the steps of method 300, any suitable combination of components, devices, or systems (including ones not explicitly disclosed) may be used to carry out the steps.

### Example SNP Genotype Inference of Some or All Genomic Contributors

In some embodiments, the system may determine the amount of contributor-derived nucleic acids by inferring the SNP genotype for some or all contributors by summarizing and grouping SNP minor allele frequency (MAF) signals, which were obtained from analyzing a panel of selected SNPs on cell-free nucleic acids from a single sample from the transplant recipient post-transplantation, or from multiple, longitudinal samples taken from the same transplant recipient over a period of time (days, weeks, months) post-transplantation.

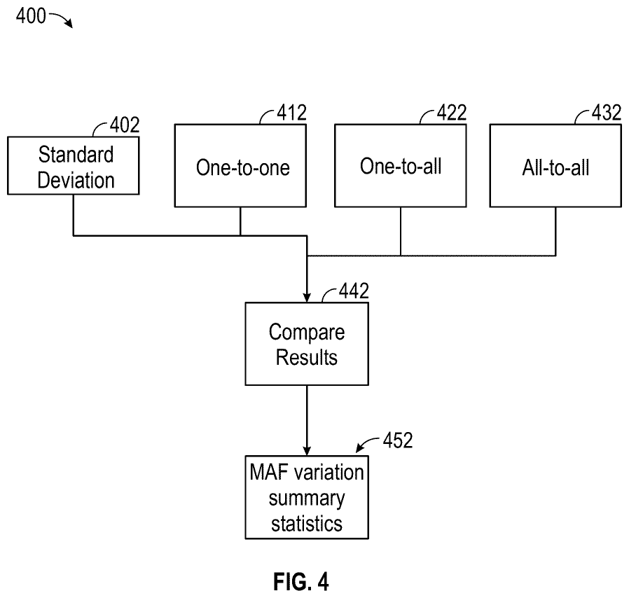

FIG. 4 illustrates an example method for grouping the SNPs and inferring the SNP genotype for some or all genomic contributors by summarizing and grouping SNP minor allele frequency (MAF) signals, which were obtained from analyzing a panel of selected SNPs on cell-free nucleic acids from multiple, longitudinal samples taken from the same transplant recipient over a period of time (days, weeks, months) post-transplantation, according to some embodiments. Method 400 may comprise determining MAF variation summary statistics, such as a simple MAF variation summary statistic of standard deviation (or min-max range) 402, “one-to-one” MAF variation summary statistics 412, “one-to-all” MAF variation summary statistics 422, and/or an “all-to-all” MAF variation summary statistics 432. In some embodiments, one or more of the processes are compared in step 442 to determine the MAF variation summary statistics 452 used in determining the amount of contributor-derived nucleic acids. In some embodiments, the processes may comprise generating one or more MAF variation summary statistics, and, in some embodiments, the step 442 comparison may involve selecting the MAF variation summary statistics that represent the greatest dissimilarity between groups amongst the one or more MAF variation summary statistics.

Simple MAF Variation Summary Statistic of Standard Deviation 402: In some embodiments, the simple MAF variation summary statistics of standard deviation (or min-max range) 402 may comprise sorting MAF values and creating a scatter plot. FIG. 5A illustrates an example waterfall plot of MAF values for a panel of SNPs for a simple MAF variation summary statistics of standard deviation, according to some embodiments. The waterfall plot may comprise a plurality of groups, such as a recipient-hetero group 502 and a recipient-homo group 504. A recipient-hetero group 502 may comprise SNPs having MAF values that may be greater than the MAF values of the SNPs of a recipient-homo group 504. As shown in the example waterfall plot, the recipient-hetero group 502 may comprise a first set of SNPs (e.g., 160 SNPs) having MAF values greater than or equal to a threshold (e.g., 0.24), and the recipient-homo group 504 may comprise the rest of the SNPs having MAF values less than the threshold (e.g., 0.24).

The waterfall plot may exhibit one or more tiers of “stairs” comprising one or more “steps” (second-tier stairs). The stairs and steps represent the groups of SNPs that share the same genotypes of one or more genomic contributors. By identifying such stairs or steps by grouping, the SNPs may be used to determine one or more genomic contributors' genotypes. For example, as shown in FIG. 5A, the first-tier stairs may be used to distinguish between a recipient-hetero group 502 and a recipient-homo group 504. In some embodiments, the first-tier stair of group 502 and group 504 may correspond to the major genomic contributor's (transplant recipient) SNP genotype, and the second-tier stairs (“steps”) may be further identified inside group 504.

In some embodiments, the waterfall plot may show a second tier of stairs (“steps”) in the recipient-homo group 504, such as shown in the plot of FIG. 5B, and one or more minor contributors' SNP genotypes may be determined from the steps. In one example, a contributor's genotype may be determined based on the stairs compared to a threshold. The determined amount of contributor-derived nucleic acids may be determined from MAF values grouped by the contributors' genotypes.

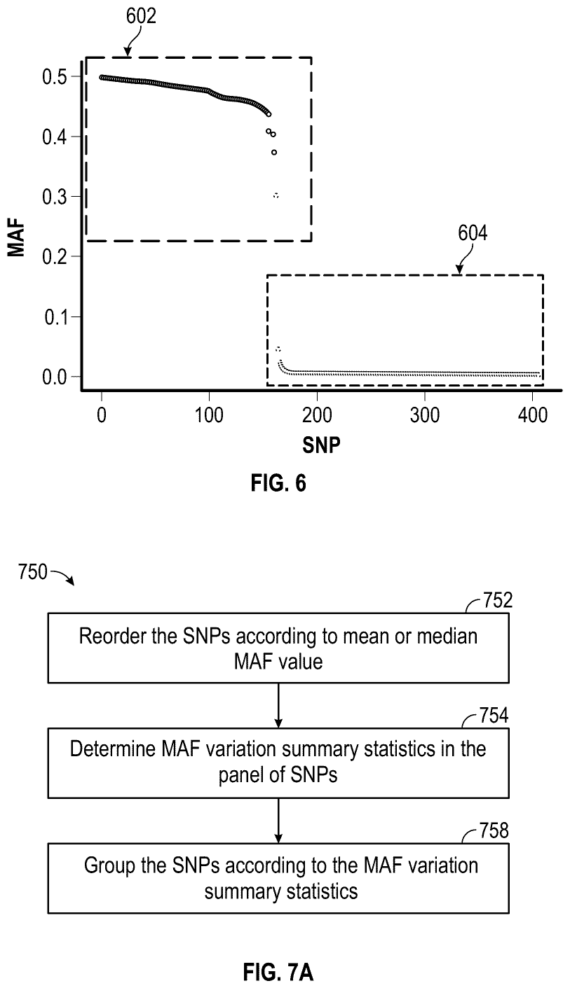

The second-tier stairs may not be discernible in the waterfall plot, such as when the SNPs are mixed. One example waterfall plot where the second-tier stairs is not discernible is shown in FIG. 6. For example, the first-tier stairs are discernible, allowing the system to separate the SNPs into a recipient-hetero group 602 and a recipient-homo group 604. In the recipient-homo group 604, the second-tier groups are mixed, and hence, may not be easily separated from each other, and as a result, the SNP genotype cannot be determined from the waterfall plot. The lack of a discernible second-tier stairs in the waterfall plot may make it difficult to accurately determine the amount of contributor-derived nucleic acids.

In some embodiments, the waterfall plot may comprise a plurality of tiers of stairs. For example, as shown in FIGS. 5A and 5B, the plurality of tiers of stairs may comprise first-tier stairs (FIG. 5A) and second-tier stairs (FIG. 5B). The first-tier stairs may be determined by separating the plot into groups based on thresholding (described above). The second-tier stairs may be determined by separating one or more first-tier stairs. In the example shown in FIG. 5B, the second-tier stairs may comprise three steps. Each step may comprise SNPs grouped together based on similar MAF values.

Embodiments of the disclosure may comprise a method for determining one or more genotypes from the MAF values. In some instances, the waterfall plot of the MAF values may not exhibit second-tier stairs. One non-limiting example is when the transplant recipient is a pregnant woman where the sample was taken in the early weeks of pregnancy (e.g., before 10 weeks since conception), before conception, or after conception. In some embodiments, the determined genotype(s) may comprise a genotype for the transplant recipient, fetus, or donor (including the previous donor(s) or the current donor) based on MAF variations, which may be determined from the MAF values.

MAF variations may be determined based on a set of longitudinal samples from the same transplant recipient, for example. The set of longitudinal samples may have the same SNP genotypes. In some embodiments, when the transplant recipient is a pregnant woman, the set of longitudinal samples may comprise one or more samples from the transplant recipient taken before conception, one or more samples from the transplant recipient taken before 10 weeks since conception, one or more samples from the transplant recipient taken after at least 10 weeks since conception, or a combination thereof. In some embodiments, when the transplant recipient has received a plurality of transplants, the set of longitudinal samples may comprise one or more samples from the transplant recipient taken before receiving one or more previous transplants, one or more samples from the transplant recipient taken before receiving the current transplant, one or more samples from the transplant recipient after receiving the previous and current transplants, or a combination thereof.

The fetal or current-donor SNP genotype may be determined from the longitudinal MAF variation. Additionally, or alternatively, a plot of the MAF variation summary statistics may be used to determine the amount of contributor-derived nucleic acids.

FIG. 7A illustrates an example method for determining MAF information in a panel of SNPs, according to some embodiments. As discussed above, the system 100 may receive nucleic acid sequence data 140. The nucleic acid sequence data 140 may comprise longitudinal MAF data (e.g., MAF values) for each SNP. Method 750 may comprise sorting the samples according to MAF values in step 752. In some embodiments, step 752 may comprise reordering the SNPs according to the mean or median MAF value of SNPs across samples. In some embodiments, the SNPs may be grouped according to homozygous or heterozygous genotype of the transplant recipient. The grouping may be performed by using threshold parameters, as discussed above.

In step 754, the system 100 may determine the MAF variation summary statistics in the panel of SNPs. The MAF variation summary statistics comprise MAF variations calculated as simple forms such as standard deviation or dynamic range or calculated as more complex forms. In some embodiments, a smoothing and differential function may be applied to the MAF variation summary statistics, resulting in an MAF-variation difference plot. FIG. 7B shows an example plot of the MAF variation summary statistics. The smoothing and differential function may be any type of function that smooths and measures the local slope of the MAF variation summary statistics. The local minimum or maximum of the smoothing and differential function may help to determine a “separation point” P in the MAF variation summary statistics. Example smoothing and differential functions may include, but are not limited to, a first derivative of a Gaussian function (using selected parameters), a linear smoothing and differential function (using a lagged difference of a sequence), or the like.

In step 758 (of FIG. 7A), the system may group the SNPs according to the MAF variation summary statistics, for example, groups 502 and 504 of FIG. 5A. This step may involve determining a separation point by determining a local minimum or maximum of the smoothing and differential function applied on the MAF variation summary statistics within a certain window of SNP location index, such as window 720 shown in FIG. 7B. The location of the window 720 may be determined based on relationship information between genomic contributors. The separation point 722 (also referred to as separation point P throughout this disclosure) may be used to group (cluster) the SNPs into homozygous and heterozygous groups. After the SNPs have been grouped, in some embodiments, the grouping may go through a final quality control check.

In some embodiments, the system may group the SNPs into more than two groups in the MAF variation summary statistics. The MAF variation summary statistics may be generated for a mixed sample of a transplant recipient who has received a plurality of transplants. FIG. 7C illustrates an example MAF waterfall plot and a plot of MAF variation summary statistics that comprise three groups of SNPs with distinct genotypes of one of the genomic contributors (transplant donor-1), according to some embodiments of the disclosure. As shown in the figure, there may be three groups 702, 704, and 706.

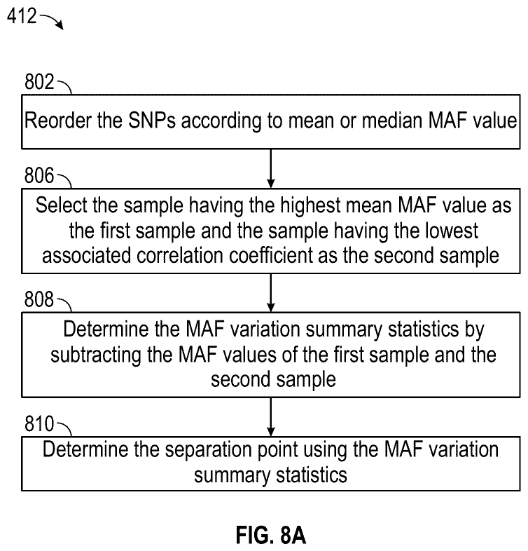

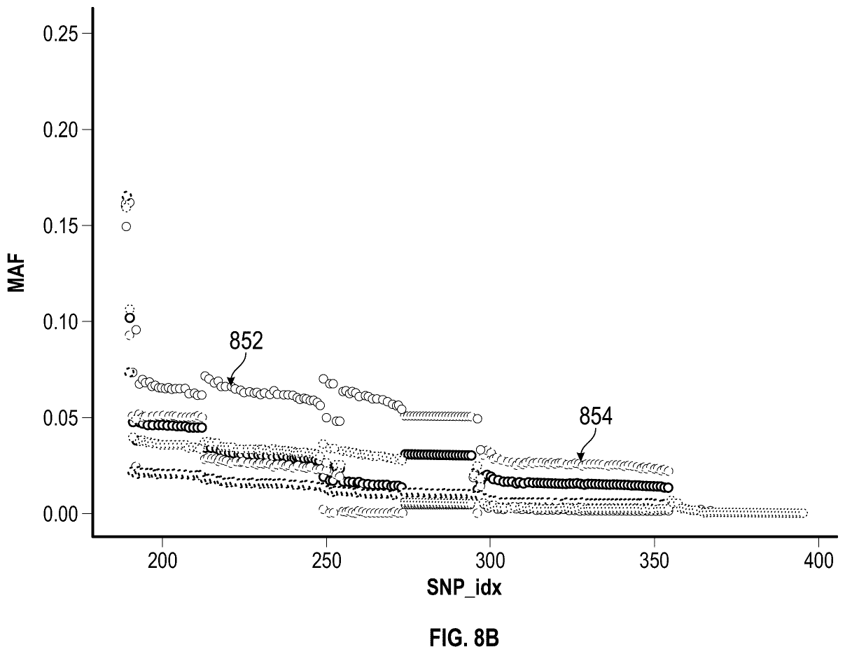

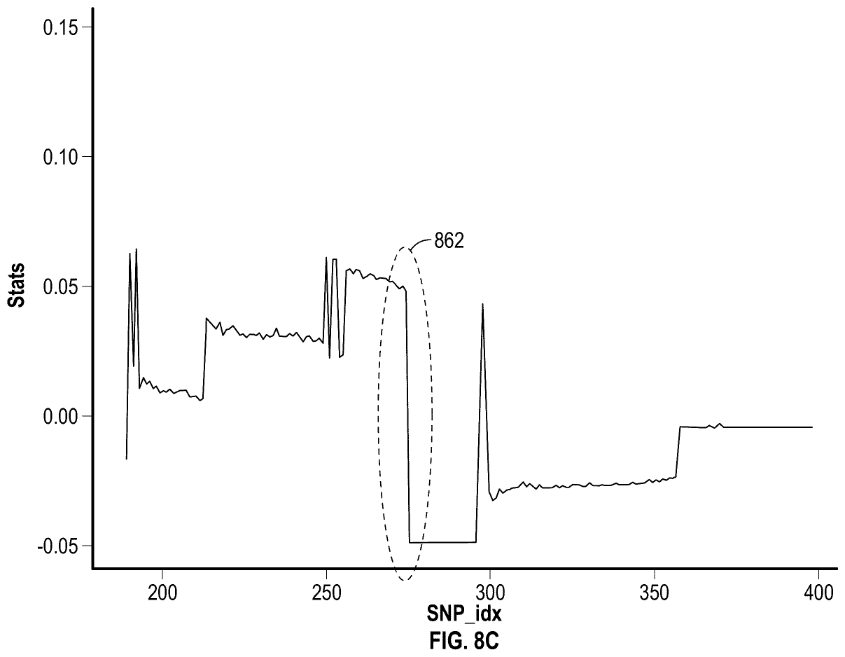

“One-To-One” MAF Variation Summary Statistics 412: The process to generate “one-to-one” MAF variation summary statistics 412 may comprise determining the sample difference of MAF values between two samples having the greatest variation of MAF values. FIG. 8A illustrates an example method for one-to-one MAF variation summary statistics, according to some embodiments. The process may comprise reordering a panel of SNPs according to mean or median MAF values (step 802). FIG. 8B illustrates example MAF waterfall plots for five samples. A sample 852 having the highest mean MAF values and a sample 854 having the lowest correlation coefficient value with sample 852 (among the plurality of samples) are selected as the first sample and second sample, respectively (step 806 of FIG. 8A). Then, in step 808, the system may subtract MAF values of the first selected sample 852 and the second selected sample 854, resulting in the MAF variation summary statistics similar to the one shown in FIG. 8C. Using this MAF variation summary statistics, in step 810 of FIG. 8A, the system may determine a separation point 862 (of FIG. 8C) on the MAF variation summary statistics (through a process similar to the one described in FIG. 7B), such as where there is a large difference (a sharp edge).

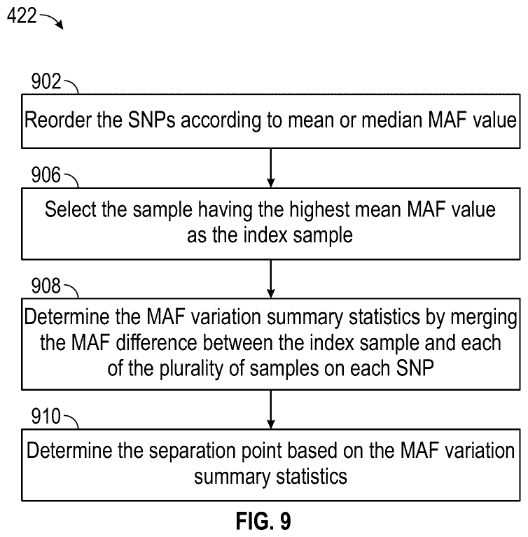

“One-To-All” MAF Variation Summary Statistics 422: In some embodiments, the process to generate “one-to-all” MAF variation summary statistics 422 may comprise determining MAF value differences of each SNP between a selected index sample and the rest of the plurality of samples. FIG. 9 illustrates an example method for one-to-all MAF variation summary statistics, according to some embodiments. Step 902 may be similar to step 802 (of process 412 in FIG. 8A). In step 906, the index sample (e.g., the first sample 852 of FIG. 8B) may be selected as the sample having the highest mean MAF value. In step 908, the MAF values of the index sample and each of the plurality of samples may be subtracted on each SNP. In some embodiments, the subtraction may comprise a subtraction from the cumulative minimum or the cumulative maximum of the MAF values of the plurality of samples. Then, as part of step 908, the resultants (from subtracting the cumulative minimum and subtracting the cumulative maximum) may be merged via a series of functions to generate the MAF variation summary statistics. In step 910, the separation point may be determined on the MAF variation summary statistics (through a process similar to the one described with respect to FIG. 7B, such as where there is a large difference (a sharp edge)).

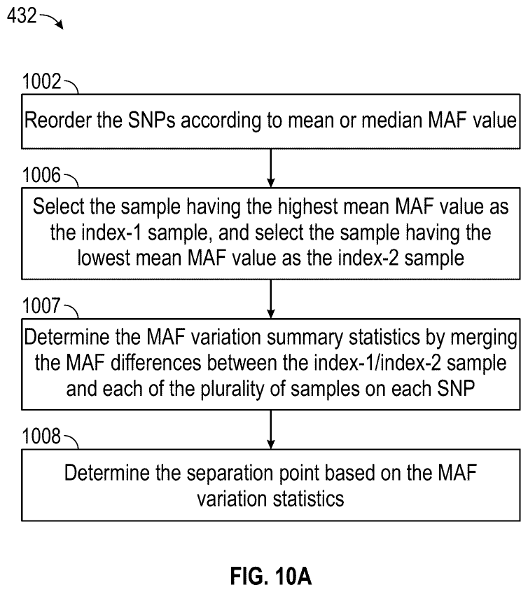

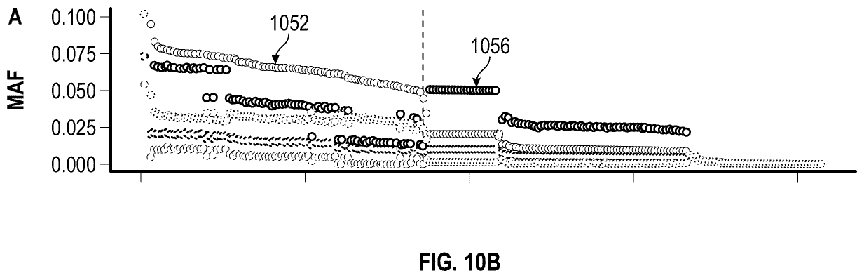

“All-To-All” MAF Variation Summary Statistics 432: The process to generate “all-to-all” MAF variation summary statistics 432 comprises determining MAF value differences of each SNP between two selected index samples and the plurality of samples. Step 1002 (of FIG. 10A) may be similar to step 802/902 of FIGS. 8A and 9, respectively. In step 1006 of the process 432, a first index sample, the index-1 sample 1052 (shown in FIG. 10B), may be selected as the sample having the highest mean MAF value of the SNPs in the higher end (a first set of a plurality of samples having higher mean MAF values), and a second index sample, the index-2 sample 1056, may be selected as the sample having the highest mean MAF value of the SNPs in the lower end (a second set of a plurality of samples having lower mean MAF values). Then for each pair of the index-1 sample and the first set of the plurality of samples, and for each pair of the index-2 sample and the second set of the plurality of samples, in step 1007, the system may subtract the MAF values and may merge them via a series of functions to generate the MAF variation summary statistics. In step 1008, the separation point may be determined on the MAF variation summary statistics through a process similar to the one described with respect to FIG. 7B, such as where there is a large difference (a sharp edge).

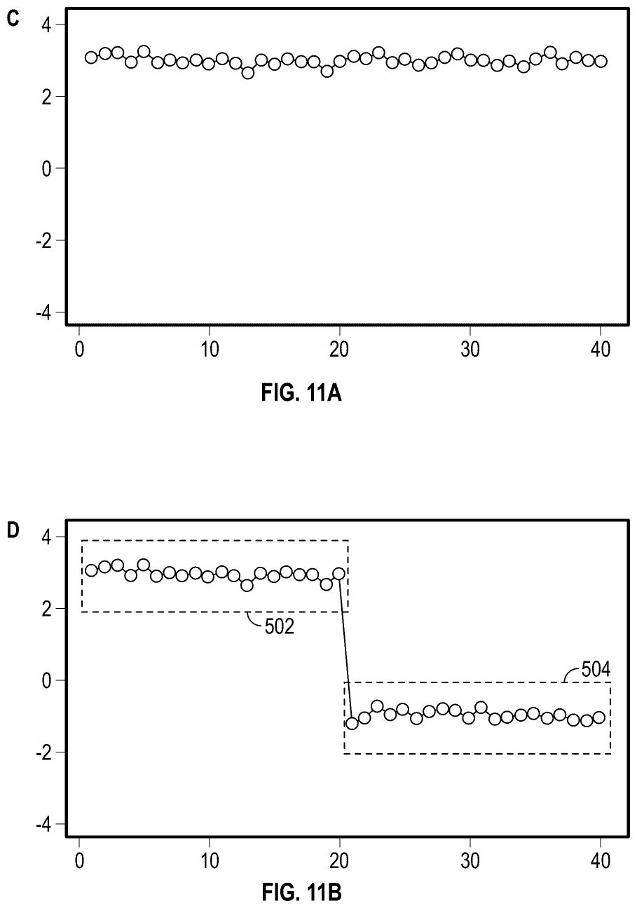

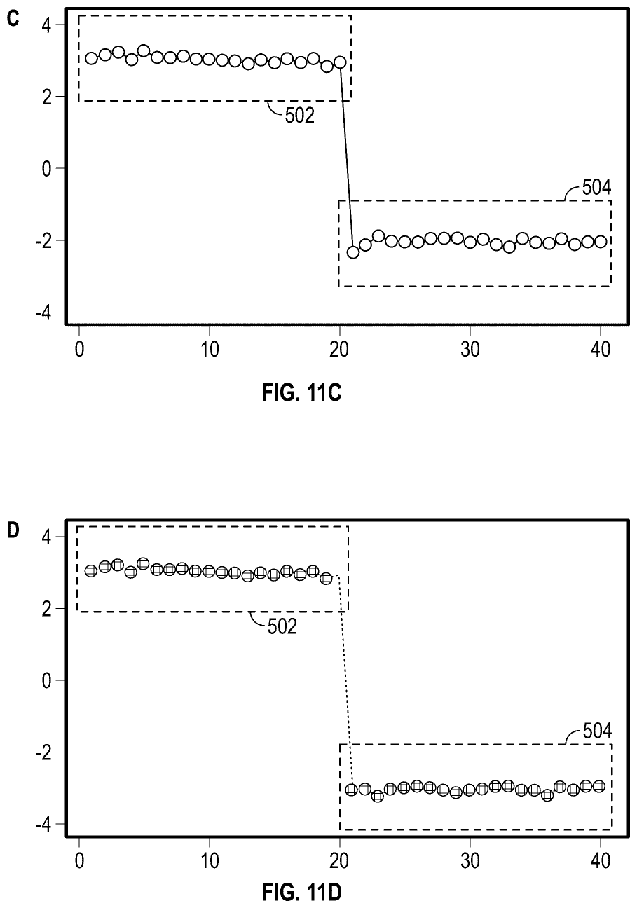

In some embodiments, results from one or more processes may be compared (e.g., step 442 of FIG. 4) to determine the MAF variation summary statistics 452. FIGS. 11A-11D illustrate example comparisons of a simple MAF variation summary statistic of standard deviation 402 (FIG. 11A), a MAF variation summary statistics (“one-to-one”) 412 (FIG. 11B), a MAF variation summary statistics (“one-to-all”) 422 (FIG. 11C), and a MAF variation summary statistics (“all-to-all”) 432 (FIG. 11D). The gaps of the MAF variation summary statistics between two groups of SNPs (502 and 504 shown in FIGS. 11B-11D) may, for example, be about 0, 4, 5, and 6 for the standard deviation, the “one-to-one” statistics, the “one-to-all” statistics, and the “all-to-all” statistics, respectively. A gap is the difference between the mean MAF values of the SNP group 502 and the SNP group 504. In some embodiments, the MAF variation summary statistics 452 may be selected as the “all-to-all” statistics 432 due to having the highest gap (of 6). In some embodiments, the waterfall plot may not have a major group and a minor group, as shown in the plot of FIG. 11A.

### Example Determination of the Amount of Contributor-Derived Cell-Free Nucleic Acids and Determination of the Risk of Transplant Rejection

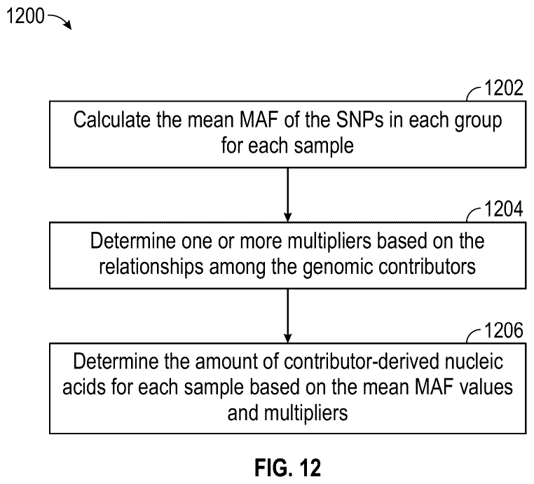

The system may determine one or more amounts of cell-free nucleic acids, such as cell-free DNA, in a mixed sample using MAF variation summary statistics. FIG. 12 illustrates an example flowchart of the method for determining the amount of contributor-derived cell-free nucleic acids, such as transplant donor-derived cell-free DNA, according to some embodiments. The method 1200 may comprise a system calculating the mean MAF information of a panel of SNPs in each group (step 1202). For example, the system may calculate a first mean MAF information for the recipient-hetero group and a second mean MAF information for the recipient-homo group.

In step 1204, the system may determine one or more multipliers based on the relationship among the genomic contributors. In some embodiments, the multiplier(s) may serve as transforming the calculated mean MAF values of grouped SNPs to the output of the amount of contributor-derived nucleic acids. For example, the multiplier may be 2 for the group of SNPs that have recipient-homo and fetus-homo genotype to yield the contributor-derived fraction, when the transplant recipient is a pregnant woman, and the donor is unrelated to the transplant recipient.

In step 1206, the system may determine the amount of contributor-derived nucleic acids. In some embodiments, the amount of contributor-derived nucleic acids may be determined based on mean MAF values and the determined multipliers. For example, each group of SNPs in the MAF variation summary statistics and corresponding mean MAF values may be associated with one or more genomic contributors. The product of the MAF values and the multipliers may result in a determined amount of cell-free DNA from the corresponding genomic contributor(s).

Embodiments of the disclosure may comprise determining a risk of transplant rejection in a transplant recipient based on the determined amount of contributor-derived nucleic acids. In some embodiments, the risk of transplant rejection is determined to be low when the determined amount of contributor-derived nucleic acids is below a predetermined threshold value, and the risk of transplant rejection is determined to be high when the estimated amount of contributor-derived nucleic acids is greater than a predetermined threshold.

### Example System for Determining the Amount of Contributor-Derived Nucleic Acids in a Mixed Sample

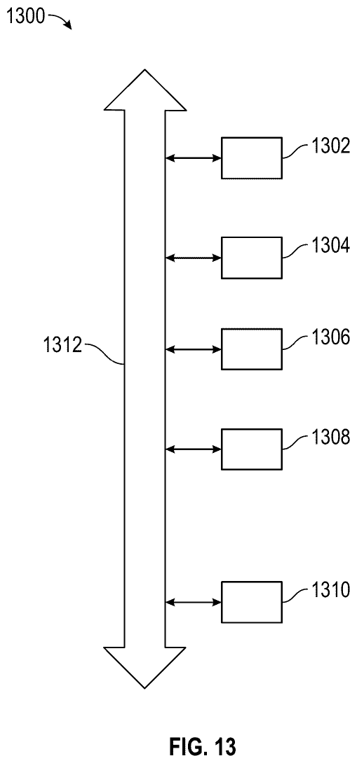

The system, kits, and methods discussed herein may be implemented by a device. FIG. 13 illustrates an example device that implements the disclosed system, kits, and methods, according to some embodiments. In some embodiments, the one or more computing device(s) 1300 may perform one or more steps of one or more methods described or illustrated herein. In certain embodiments, the one or more computing device(s) 1300 may provide functionality described or illustrated herein. In certain embodiments, software running on the one or more computing device(s) 1300 may perform one or more steps of one or more methods described or illustrated herein or may provide functionality described or illustrated herein. Certain embodiments may include one or more portions of the one or more computing device(s) 1300.

This disclosure contemplates any suitable number of computing systems 1300. This disclosure contemplates one or more computing device(s) 1300 taking any suitable physical form. As example and not by way of limitation, one or more computing device(s) 1300 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, the one or more computing device(s) 1300 may be unitary or distributed; span multiple locations; span multiple machines; span multiple data centers; or reside in a cloud, which may include one or more cloud components in one or more networks.

Where appropriate, the one or more computing device(s) 1300 may perform without substantial spatial or temporal limitation one or more steps of one or more methods described or illustrated herein. As an example, and not by way of limitation, the one or more computing device(s) 1300 may perform in real time or in batch mode one or more steps of one or more methods described or illustrated herein. The one or more computing device(s) 1300 may perform at different times or at different locations one or more steps of one or more methods described or illustrated herein, where appropriate.

In certain embodiments, the one or more computing device(s) 1300 may include a processor 1302, memory 1304, database 1306, an input/output (I/O) interface 1308, a communication interface 1310, and a bus 1312. Although this disclosure describes and illustrates a particular computer system having a particular number of particular components in a particular arrangement, this disclosure contemplates any suitable computer system having any suitable number of any suitable components in any suitable arrangement. In certain embodiments, processor 1302 may include hardware for executing instructions, such as those making up a computer program. As an example, and not by way of limitation, to execute instructions, processor 1302 may retrieve (or fetch) the instructions from an internal register, an internal cache, memory 1304, or database 1306; decode and execute them; and then write one or more results to an internal register, an internal cache, memory 1304, or database 1306. In certain embodiments, processor 1302 may include one or more internal caches for data, instructions, or addresses. This disclosure contemplates processor 1302 including any suitable number of any suitable internal caches, where appropriate. As an example, and not by way of limitation, processor 1302 may include one or more instruction caches, one or more data caches, and one or more translation lookaside buffers (TLBs). Instructions in the instruction caches may be copies of instructions in memory 1304 or database 1306, and the instruction caches may speed up retrieval of those instructions by processor 1302.

Data in the data caches may be copies of data in memory 1304 or database 1306 for instructions executing at processor 1302 to operate on; the results of previous instructions executed at processor 1302 for access by subsequent instructions executing at processor 1302 or for writing to memory 1304 or database 1306; or other suitable data. The data caches may speed up read or write operations by processor 1302. The TLBs may speed up virtual-address translation for processor 1302. In certain embodiments, processor 1302 may include one or more internal registers for data, instructions, or addresses. This disclosure contemplates processor 1302, including any suitable number of any suitable internal registers, where appropriate. Where appropriate, processor 1302 may include one or more arithmetic logic units (ALUs); be a multi-core processor; or include one or more processors 1302. Although this disclosure describes and illustrates a particular processor, this disclosure contemplates any suitable processor.

In certain embodiments, memory 1304 includes main memory for storing instructions for processor 1302 to execute data for processor 1302 to operate on. As an example, and not by way of limitation, the one or more computing device(s) 1300 may load instructions from database 1306 or another source (such as, for example, another one or more computing device(s) 1300) to memory 1304. Processor 1302 may then load the instructions from memory 1304 to an internal register or internal cache. To execute the instructions, processor 1302 may retrieve the instructions from the internal register or internal cache and decode them. During or after execution of the instructions, processor 1302 may write one or more results (which may be intermediate or final results) to the internal register or internal cache. Processor 1302 may then write one or more of those results to memory 1304.

In certain embodiments, processor 1302 executes only instructions in one or more internal registers or internal caches or in memory 1304 (as opposed to database 1306 or elsewhere) and operates only on data in one or more internal registers or internal caches or in memory 1304 (as opposed to database 1306 or elsewhere). One or more memory buses (which may each include an address bus and a data bus) may couple processor 1302 to memory 1304. Bus 1312 may include one or more memory buses, as described below. In certain embodiments, one or more memory management units (MMUs) reside between processor 1302 and memory 1304 and facilitate accesses to memory 1304 requested by processor 1302. In certain embodiments, memory 1304 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1304 may include one or more memory devices, where appropriate. Although this disclosure describes and illustrates particular memory, this disclosure contemplates any suitable memory.

In certain embodiments, database 1306 includes mass storage for data or instructions. As an example, and not by way of limitation, database 1306 may include a hard disk drive (HDD), a floppy disk drive, flash memory, an optical disc, a magneto-optical disc, magnetic tape, or a Universal Serial Bus (USB) drive or a combination of two or more of these. Database 1306 may include removable or non-removable (or fixed) media, where appropriate. Database 1306 may be internal or external to the one or more computing device(s) 1300, where appropriate. In certain embodiments, database 1306 is non-volatile, solid-state memory. In certain embodiments, database 1306 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass database 1306 taking any suitable physical form. Database 1306 may include one or more storage control units facilitating communication between processor 1302 and database 1306, where appropriate. Where appropriate, database 1306 may include one or more databases 1306. Although this disclosure describes and illustrates particular storage, this disclosure contemplates any suitable storage.

In certain embodiments, I/O interface 1308 includes hardware, software, or both, providing one or more interfaces for communication between the one or more computing device(s) 1300 and one or more I/O devices. The one or more computing device(s) 1300 may include one or more of these I/O devices, where appropriate. One or more of these I/O devices may enable communication between a person and the one or more computing device(s) 1300. As an example, and not by way of limitation, an I/O device may include a keyboard, keypad, microphone, monitor, mouse, printer, scanner, speaker, still camera, stylus, tablet, touch screen, trackball, video camera, another suitable I/O device, or a combination of two or more of these. An I/O device may include one or more sensors. This disclosure contemplates any suitable I/O devices and any suitable I/O interfaces 1308 for them. Where appropriate, I/O interface 1308 may include one or more device or software drivers enabling processor 1302 to drive one or more of these I/O devices. I/O interface 1308 may include one or more I/O interfaces 1308, where appropriate. Although this disclosure describes and illustrates a particular I/O interface, this disclosure contemplates any suitable I/O interface.

In certain embodiments, communication interface 1310 includes hardware, software, or both providing one or more interfaces for communication (such as, for example, packet-based communication) between the one or more computing device(s) 1300 and one or more other computing device(s) 1300 or one or more networks. As an example, and not by way of limitation, communication interface 1310 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1310 for it.

As an example, and not by way of limitation, the one or more computing device(s) 1300 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the one or more computing device(s) 1300 may communicate with a wireless PAN (WPAN) (such as, for example, a Bluetooth WPAN), a WI-FI network, a WI-MAX network, a cellular telephone network (such as, for example, a Global System for Mobile Communications (GSM) network), or other suitable wireless network or a combination of two or more of these. The one or more computing device(s) 1300 may include any suitable communication interface 1310 for any of these networks, where appropriate. Communication interface 1310 may include one or more communication interfaces 1310, where appropriate. Although this disclosure describes and illustrates a particular communication interface, this disclosure contemplates any suitable communication interface.

In certain embodiments, bus 1312 includes hardware, software, or both coupling components of the one or more computing device(s) 1300 to each other. As an example, and not by way of limitation, bus 1312 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1312 may include one or more buses 1312, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such as, for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

The term computer-readable non-transitory storage medium or media may include a single medium or multiple media (e.g., a centralized or distributed database and/or associated caches and servers) that store the one or more sets of instructions. The term “computer-readable non-transitory storage medium” shall also be taken to include any medium that is capable of storing, encoding, or carrying a set of instructions for execution by the device and that causes the device to perform any one or more of the methods of the present invention. The term “computer-readable non-transitory storage medium” shall accordingly be taken to include, but not be limited to, solid-state memories, optical and magnetic media, and carrier wave signals.

Although examples of this disclosure have been fully described with reference to the accompanying drawings, it is to be noted that various changes and modifications will become apparent to those skilled in the art. Such changes and modifications are to be understood as being included within the scope of examples of this disclosure as defined by the appended claims.

1. A computer-implemented method of outputting an amount of contributor-derived nucleic acids in a mixed sample, obtained from a transplant recipient who has received at least two transplants, comprising nucleic acids from at least three genetically distinct contributors, the method comprising:
receiving, via a computer or an input function, nucleic acid sequence data from a panel of single nucleotide polymorphisms (SNPs) from the nucleic acids from the at least three genetically distinct contributors, wherein the at least three genetically distinct contributors comprise a recipient genomic contributor, a first transplant donor genomic contributor, and a second transplant donor genomic contributor;
receiving a genomic relationship among the at least three genetically distinct contributors;
determining and grouping minor allele frequency (MAF) information from the panel of SNPs; and
outputting an amount of contributor-derived nucleic acids based on the genomic relationship and the MAF information grouping.

2. The computer-implemented method of claim 1, wherein determining and grouping MAF information are based on a set of longitudinal samples.

3. The computer-implemented method of claim 2, wherein the set of longitudinal samples have the same genotype.

4. The computer-implemented method of claim 1, wherein the panel of SNPs comprises fewer than 500 SNPs.

5. The computer-implemented method of claim 1, further comprising:
determining a genotype of one or more of genomic contributors based on the MAF information grouping.

6. The computer-implemented method of claim 1, wherein determining and grouping MAF information comprises:
reordering the panel of SNPs according to mean or median MAF value;
determining the MAF information comprising MAF variation summary statistics in the panel of SNPs; and
grouping the panel of SNPs according to the MAF variation summary statistics.

7. The computer-implemented method of claim 6, wherein determining and grouping MAF information comprises:
determining a separation point in the MAF variation summary statistics by determining a local minimum or maximum in a window.

8. The computer-implemented method of claim 7, wherein the separation point is used to group the SNPs into homozygous and heterozygous genotype groups.

9. The computer-implemented method of claim 1, wherein determining and grouping MAF information comprises:
generating a waterfall plot of the MAF information;
grouping the MAF information by segmenting the waterfall plot into groups; and
calculating mean MAF values of the groups, wherein the determined amount of contributor-derived nucleic acids is based on the calculated mean MAF values.

10. The computer-implemented method of claim 1, wherein determining and grouping MAF information comprises:
selecting a first sample comprising a highest mean MAF value among a plurality of samples;
selecting a second sample comprising a lowest correlation coefficient associated with the first sample;
determining MAF variation summary statistics by subtracting MAF values of the selected first sample and the selected second sample;
determining a separation point in the MAF variation summary statistics; and
grouping the MAF information based on the separation point.

11. The computer-implemented method of claim 1, wherein determining and grouping MAF information comprises:
selecting an index sample comprising a highest mean MAF value among a plurality of samples;
determining an MAF difference between the index sample and each of the plurality of samples;
determining MAF variation summary statistics by merging the MAF differences;
determining a separation point in the MAF variation summary statistics; and
grouping the MAF information based on the separation point.

12. The computer-implemented method of claim 1, wherein determining and grouping MAF information comprises:
selecting a first index sample comprising a highest mean MAF value among a set of high reordered SNPs;
selecting a second index sample comprising a highest mean MAF among a set of low reordered SNPs;
determining an MAF difference between the first index sample and each of the set of high reordered SNPs;
determining an MAF difference between the second index sample and each of the set of low reordered SNPs;
determining MAF variation summary statistics by merging the MAF differences;
determining a separation point in the MAF variation summary statistics; and
grouping the MAF information based on the separation point.

13. The computer-implemented method of claim 1, further comprising:
generating a waterfall plot for the mixed sample, wherein the waterfall plot comprises one or more tiers of stairs having one or more steps, and the SNPs of the one or more steps have the same genotype.

14. The computer-implemented method of any of claim 1, wherein the transplant recipient received a transplant comprising one or more of: a kidney transplant, a heart transplant, a lung transplant, a liver transplant, a pancreas transplant, a vascularized composite transplant, an intestinal transplant, a stomach transplant, a testis transplant, a penis transplant, an ovary transplant, a uterus transplant, a thymus transplant, a face transplant, a hand transplant, a leg transplant, a bone transplant, a cornea transplant, skin transplant, a heart valve transplant, a blood vessel transplant, or any combination thereof.

15. The computer-implemented method of claim 1, wherein the mixed sample is a blood sample.

16. A system for outputting an amount of contributor-derived nucleic acids in a mixed sample, obtained from a transplant recipient who has received at least two transplants, comprising nucleic acids from at least three genetically distinct contributors, the system comprising:
an interface configured to receive an input;
a determination unit configured to:
receive, via the interface, nucleic acid sequence data from a panel of single nucleotide polymorphisms (SNPs) from the nucleic acids from the at least three genetically distinct contributors, wherein the at least three genetically distinct contributors comprise a recipient genomic contributor, a first transplant donor genomic contributor, and a second transplant donor genomic contributor;
receive a genomic relationship among the at least three genetically distinct contributors;
determine and group minor allele frequency (MAF) information from the panel of SNPs; and
output an amount of contributor-derived nucleic acids based on the genomic relationship and the MAF information grouping.

17. The system of claim 16, wherein determining and group MAF information are based on a set of longitudinal samples.

18. The system of claim 17, wherein the set of longitudinal samples have the same genotype.

19. The system of claim 16, wherein the panel of SNPs comprises fewer than 500 SNPs.

20. The system of claim 16, wherein the determination unit is further configured to:
determine a genotype of one or more of genomic contributors based on the MAF information grouping.

21. The system of claim 16, wherein the determination unit configured to determine and group MAF information comprises the determination unit configured to:
reorder the panel of SNPs according to mean or median MAF value;
determine the MAF information comprising MAF variation summary statistics in the panel of SNPs; and
group the panel of SNPs according to the MAF variation summary statistics.

22. The system of claim 21, wherein the determination unit configured to determine and group MAF information comprises the determination unit configured to:
determine a separation point in the MAF variation summary statistics by determining a local minimum or maximum in a window.

23. The system of claim 22, wherein the separation point is used to group the SNPs into homozygous and heterozygous genotype groups.

24. The system of claim 16, wherein the determination unit configured to determine and group MAF information comprises the determination unit configured to:
generate a waterfall plot of the MAF information;
group the MAF information by segmenting the waterfall plot into groups; and
calculate mean MAF values of the groups, wherein the determined amount of contributor-derived nucleic acids is based on the calculated mean MAF values.

25. The system of claim 16, wherein the determination unit configured to determine and group MAF information comprises the determination unit configured to:
select a first sample comprising a highest mean MAF value among a plurality of samples;
select a second sample comprising a lowest correlation coefficient associated with the first sample;
determine MAF variation summary statistics by subtracting MAF values of the selected first sample and the selected second sample;
determine a separation point in the MAF variation summary statistics; and
group the MAF information based on the separation point.

26. The system of claim 16, wherein the determination unit configured to determine and group MAF information comprises the determination unit configured to:
select an index sample comprising a highest mean MAF value among a plurality of samples;
determine an MAF difference between the index sample and each of the plurality of samples;
determine MAF variation summary statistics by merging the MAF differences;
determine a separation point in the MAF variation summary statistics; and
group the MAF information based on the separation point.

27. The system of claim 16, wherein the determination unit configured to determine and group MAF information comprises the determination unit configured to:
select a first index sample comprising a highest mean MAF value among a set of high reordered SNPs;
select a second index sample comprising a highest mean MAF among a set of low reordered SNPs;
determine an MAF difference between the first index sample and each of the set of high reordered SNPs;
determine an MAF difference between the second index sample and each of the set of low reordered SNPs;
determine MAF variation summary statistics by merging the MAF differences;
determine a separation point in the MAF variation summary statistics; and
group the MAF information based on the separation point.

28. The system of claim 16, wherein the determination unit is further configured to:
generate a waterfall plot for the mixed sample, wherein the waterfall plot comprises one or more tiers of stairs having one or more steps, and the SNPs of the one or more steps have the same genotype.

29. The system of any claim 16, wherein the transplant recipient received a transplant comprising one or more of: a kidney transplant, a heart transplant, a lung transplant, a liver transplant, a pancreas transplant, a vascularized composite transplant, an intestinal transplant, a stomach transplant, a testis transplant, a penis transplant, an ovary transplant, a uterus transplant, a thymus transplant, a face transplant, a hand transplant, a leg transplant, a bone transplant, a cornea transplant, skin transplant, a heart valve transplant, a blood vessel transplant, or any combination thereof.

30. The system of claim 16, wherein the mixed sample is a blood sample.
